SEQUENCE LISTING (7 sequences)

SEQ ID NO: 1 - DNA, 20 bp, Artificial Sequence
  sgRNA targeting sequence
gctgaactggtcgcagttga

SEQ ID NO: 2 - DNA, 20 bp, Artificial Sequence
  sgRNA targeting sequence
gatattggcataagcctccc

SEQ ID NO: 3 - DNA, 21 bp, Artificial Sequence
  sgRNA targeting sequence
gtcaactgcgaccagttcagc

SEQ ID NO: 4 - DNA, 19 bp, Artificial Sequence
  sgRNA targeting sequence
tggttgctgggagcgacgc

SEQ ID NO: 5 - DNA, 19 bp, Artificial Sequence
  primer
caccagccctctttccaga

SEQ ID NO: 6 - DNA, 20 bp, Artificial Sequence
  primer
ccccttgcaatgatttctgt

SEQ ID NO: 7 - RNA, 21 nt, Artificial Sequence
  siRNA
gaagugaucggugagaguauu

(19) United States
(12) Patent Application Publication
(10) Pub. No.: US 2018/0066253 A1
(43) Pub. Date: Mar. 8, 2018

(54) METHODS AND COMPOSITIONS FOR MODIFYING ENDOTHELIAL CELLS

(71) Applicant: YALE UNIVERSITY, New Haven, CT (US)

(72) Inventors: Jordan POBER, Guilford, CT (US); Parwiz ABRAHIMI, New Haven, CT (US)

(21) Appl. No.: 15/560,007

(22) PCT Filed: Mar. 22, 2016
(86) PCT No.: PCT/US16/23523

§ 371 Date: Sep. 20, 2017

Related U.S. Application Data
(60) Provisional application No. 62/138,440, filed on Mar. 26, 2015.

(51) Int. Cl. C12N 15/113; C12N 9/22; C12N 15/11; A61K 35/44

(52) U.S. Cl. CPC C12N 15/113; C12N 9/22; C12N 2310/20; A61K 35/44; C12N 2310/14; C12N 15/11

(57) ABSTRACT

The present invention includes methods and compositions for modifying endothelial cells for improved vascular self-assembly. In some embodiments, the invention includes a modified endothelial cell and methods of generating and using the modified endothelial cell in a pharmaceutical composition. Other embodiments include methods of promoting vascular self-assembly and regenerating vascular structures in a subject in need thereof. In particular, the modified endothelial cell has reduced immunogenicity in an allogeneic environment in the subject, while still capable of forming vascular structures.

### CROSS-REFERENCE TO RELATED APPLICATIONS

The present application claims priority under 35 U.S.C. § 119(e) to U.S. Provisional Patent Application No. 62/138,440, filed Mar. 26, 2015, which application is incorporated herein by reference in its entirety.

### STATEMENT REGARDING FEDERALLY SPONSORED RESEARCH OR DEVELOPMENT

This invention was made with government support under HL051014, HL085416, HL109455 and AI112218 awarded by the National Institute of Health. The government has certain rights in the invention.

### BACKGROUND OF THE INVENTION

Endothelial cells (EC) are critical participants in and regulators of numerous processes including inflammation, immunity, wound healing, coagulation, fibrinolysis, macromolecular transport, permselectivity and organ perfusion. ECs form the inner lining of blood vessels and provides a barrier between the vessel wall and blood. They react with physical and chemical stimuli within the circulation and regulate hemostasis, vasomotor tone, and immune and inflammatory responses. In addition, ECs are pivotal in angiogenesis and vasculogenesis. Injury, activation or dysfunction is a hallmark of many pathologic states including atherosclerosis, loss of semi-permeable membrane function, and thrombosis.

Animal models have offered important insights into EC biology, but cultured EC are widely used to dissect processes that are difficult to analyze in whole-animal studies. The majority of these in vitro experiments use untransformed human EC cultures, the most common system being human umbilical vein endothelial cells (HUVEC). Cultured human EC offer two clear advantages over using cultured mouse EC: (a) they can be serially passaged without transformation, thereby avoiding a process that frequently alters their phenotypes, and (b) their properties differ from those in mouse EC thereby making study results more applicable to human biology and disease. For example, human EC can activate alloantigen-reactive memory CD4⁺ T cells to elicit effector functions, a property requiring both expression of class II MHC molecules and the expression of CD58 (also known as LFA-3), a major positive co-stimulatory molecule not found in mice. In contrast, mouse EC, lacking CD58, only activate CD4⁺ regulatory T cells, leading to very different outcomes regarding the roles played by EC in transplantation. Specifically, the ability of human EC to activate effector memory CD4⁺ T cells in vivo can explain why cell-mediated rejection of vascularized human allografts can occur despite deletion of professional antigen presenting cells (“passenger leukocytes”) whereas mouse grafts are significantly protected by the same approach. These immunological functions of human EC are also a concern for the immune response to tissue-engineered grafts constructed from allogeneic sources of cells.

Human endothelial cells spontaneously self-assemble into blood vessels, a property that is desirable for repair of injured organs or for establishing perfusion of engineered replacement tissues such as skin. However, human endothelial cells also have the capacity to recruit and activate memory T cells, initiating rejection of allogeneic cells and tissues. Despite the importance of evaluating EC functions with untransformed human EC cultures, they are typically hard-to-transfect, have a limited replicative lifespan and are not amenable to cloning after stable genetic manipulation.

Therefore a need exists for more effective methods of modifying endothelial cells, while maintaining replicative lifespan and stable genetic manipulation.

### SUMMARY OF THE INVENTION

As described below, the present invention includes methods and compositions for modifying endothelial cells for improved vascular self-assembly.

In one aspect, the invention includes a modified endothelial cell comprising a nucleic acid capable of downregulating gene expression of a gene selected from the group consisting of a class I and class II major histocompatibility complex (MHC) molecule, CD58, CIITA, NRLC5, beta2 microglobulin, ICOS-ligand, 4-1BB ligand, O×40 ligand, GITR ligand, interleukin 1 alpha, interleukin 6, ICAM-1, ICAM-2, VCAM-1, E-selectin, P-selectin and combinations thereof, and wherein the modified endothelial cell has reduced immunogenicity in an allogeneic environment.

In another aspect, the invention includes a modified endothelial cell comprising an inducible Cas9 expression vector; a single guide RNA specific for CD58; and a single guide RNA specific for CIITA, wherein induction of Cas9 expression results in the cell having reduced immunogenicity in an allogeneic environment.

In various embodiments of the above aspects or any other aspect of the invention delineated herein, the nucleic acid capable of downregulating gene expression is selected from the group consisting of a siRNA and a CRISPR system. In some embodiments with a siRNA, the siRNA comprises SEQ ID NO:7.

In some embodiments with a CRISPR system, the CRISPR system further comprises a Cas expression vector and a guide nucleic acid sequence specific for the gene. In another embodiment, the guide nucleic acid sequence comprising a single guide RNA, such as the single guide RNA comprising at least one sequence selected from the group consisting of SEQ ID NO: 1, SEQ ID NO: 2, SEQ ID NO: 3, and SEQ ID NO: 4. In yet another embodiment, the Cas expression vector is under the control of an inducible promoter. In still another embodiment, the Cas expression vector is a viral vector selected from the group consisting of a Sendai viral vector, an adenoviral vector, an adeno-associated viral vector, a retroviral vector, and a lentiviral vector.

In yet another aspect, the invention includes a method for generating a modified endothelial cell comprising culturing endothelial colony forming cells (ECFCs); and introducing a nucleic acid capable of downregulating gene expression of an endogenous gene selected from the group consisting of a class I and class II major histocompatibility complex (MEW) molecule, CD58, CIITA, NRLC5, beta2 microglobulin, ICOS-ligand, 4-1BB ligand, O×40 ligand, GITR ligand, interleukin 1 alpha, interleukin 6, ICAM-1, ICAM-2, VCAM-1, E-selectin, P-selectin and combinations thereof in the ECFCs, wherein the modified endothelial cell has reduced immunogenicity in an allogeneic environment.

In still another aspect, the invention includes a use of the modified endothelial cell described herein in the manufacture of a medicament for regenerating vascular structures in a subject in need thereof.

In another aspect, the invention includes a use of the modified endothelial cell described herein in the manufacture of an engineered tissue or organ.

In various embodiments of the above aspects or any other aspect of the invention delineated herein, the ECFCs are obtained from cord blood endothelial progenitor cells. In another embodiment, culturing the ECFCs comprises differentiating cord blood mononuclear cells.

In yet another embodiment, introducing the nucleic acid further comprises introducing a nucleic acid capable of downregulating gene expression is selected from the group consisting of a siRNA and a CRISPR system. In some embodiments with a siRNA, the siRNA comprises SEQ ID NO:7.

In some embodiments with a CRISPR system, the CRISPR system comprises a Cas expression vector and a guide nucleic acid sequence specific for a gene, and introducing the nucleic acid comprises introducing the Cas expression vector and the guide nucleic acid into the ECFCs; and inducing Cas expression from the Cas expression vector and, wherein the expressed Cas interacts with the guide nucleic acid sequence to mutate a loci for the gene. In another embodiment, introducing the Cas expression vector comprises transducing the ECFCs with a viral Cas expression vector, such as the viral Cas expression vector selected from the group consisting of a Sendai viral vector, an adenoviral vector, an adeno-associated viral vector, a retroviral vector, and a lentiviral vector. In still another embodiment, inducing the Cas expression vector comprises exposing the ECFCs to an agent that activates an inducible promoter in the Cas expression vector. In another embodiment, the guide nucleic acid sequence is a single guide RNA, such as the single guide RNA comprising at least one sequence selected from the group consisting of SEQ ID NO: 1, SEQ ID NO: 2, SEQ ID NO: 3, and SEQ ID NO: 4.

In yet another aspect, the invention includes a pharmaceutical composition comprising the modified endothelial cell generated according to the method described herein and a pharmaceutically acceptable carrier.

In still another aspect, the invention includes an engineered tissue or organ comprising the modified endothelial cell described herein.

In another aspect, the invention includes a method for promoting vascular self-assembly in a subject in need thereof comprising administering to a subject an effective amount of a modified endothelial cell capable of forming vascular structures, wherein the modified endothelial cell comprises a nucleic acid capable of downregulating gene expression of an endogenous gene selected from the group consisting of a class I and class II major histocompatibility complex (MHC) molecule, CD58, CIITA, NRLC5, beta2 microglobulin, ICOS-ligand, 4-1BB ligand, O×40 ligand, GITR ligand, interleukin 1 alpha, interleukin 6, ICAM-1, ICAM-2, VCAM-1, E-selectin, P-selectin and combinations thereof, and wherein the modified endothelial cell has reduced immunogenicity in an allogeneic environment while maintaining the capacity to form vascular structures.

In yet another aspect, the invention includes a method for regenerating vascular structures in a subject in need thereof comprising administering to a subject an effective amount of a modified endothelial cell capable of forming vascular structures, wherein the modified endothelial cell comprises an inducible Cas9 expression vector; a guide nucleic acid sequence specific for CD58; and a guide nucleic acid sequence specific for CIITA, and wherein induction of Cas9 expression results in the modified endothelial cell having reduced immunogenicity in an allogeneic environment while maintaining the capacity to form vascular structures.

In various embodiments of the above aspects or any other aspect of the invention delineated herein, the nucleic acid capable of downregulating gene expression is selected from the group consisting of a siRNA and a CRISPR system. In some embodiments with a siRNA, the siRNA comprises SEQ ID NO:7.

In some embodiments with a CRISPR system, the CRISPR system further comprises a Cas expression vector and a guide nucleic acid sequence specific for the gene. In another embodiment, the guide nucleic acid sequence comprising a single guide RNA, such as the single guide RNA comprising at least one sequence selected from the group consisting of SEQ ID NO: 1, SEQ ID NO: 2, SEQ ID NO: 3, and SEQ ID NO: 4. In yet another embodiment, the Cas expression vector is under the control of an inducible promoter. In still another embodiment, the Cas expression vector is a viral vector selected from the group consisting of a Sendai viral vector, an adenoviral vector, an adeno-associated viral vector, a retroviral vector, and a lentiviral vector.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A is a panel of graphs showing the high efficiency disruption of CIITA by CRISPR/Cas9 in human EC. Unmodified EC stimulated with IFN-γincreased expression of MHC class I (HLA-A,B,C) and class II (HLA-DR), whereas a high proportion of EC transduced with TetOn-Cas9/sgCIITA vectors upregulated MHC class I but showed either reduced (HLA-DR^{mid}) or complete loss of MHC class II (HLA-DR^{neg}) upregulation.

FIG. 1B is a panel of images showing the disruption of CIITA by CRISPR/Cas9 in human EC. HLA-DR^{neg} EC were clonally sorted by single-cell FACS, expanded, and sequenced across sgRNA target site to demonstrate bi-allelic indel mutations in CIITA. Representative plots shown from multiple donors from 3 independent experiments.

FIG. 2A is a panel of graphs showing that CIITA^{null} EC retained their characteristic endothelial identity. FACS isolated CIITA^{null} EC expanded for two weeks remained refractory to IFN-γ-induced upregulation of MHC II but were otherwise indistinguishable from unmodified (WT) EC with respect to surface marker expression (PECAM-1, blood group H antigen).

FIG. 2B is a graph showing viability of CIITA^{null} EC.

FIG. 2C is a panel of images showing formation of VE-cadherin positive cell-cell lateral borders.

FIG. 2D is a panel of graphs showing the characteristic TNF-α+IFN- γ responses of CIITA^{null} EC. Representative results from 3 independent donors. Scale bars 50 μm

FIG. 3A is a set of images illustrating that CIITA^{null} EC retained the ability to form vessels in vitro. CIITA^{null} EC, like unmodified (WT) EC, spontaneously assembled into cord-like structures (outlined) in 3-D culture in vitro at 24 h. Representative figures and mean±SD from 4 gels. Scale bar 25 μM.

FIG. 3B is a panel of illustrations showing CIITA^{null} EC formed perfused vessels (arrows) when implanted in SCID/bg mice like unmodified WT EC as detected by H&E. Representative figures and mean±SD from 9 mice bearing WT and CIITA^{null} implants. Scale bar 50 μM.

FIG. 3C is a panel of illustrations showing that perfused vessels formed from CIITA^{null} EC were refractory to IFN-γ induced expression of MHC II in vivo as detected by immunofluorescence. Representative figures and mean±SD from 9 mice bearing WT and CIITA^{null} implants. Scale bar 50 μM.

FIG. 4A is a panel of graphs showing loss of the ability of CIITA^{null} EC to activate allogeneic CD4+ memory T cells rescued by CIITA transduction. CIITA^{null} EC lost the ability to activate alloreactive CD4+ cells as measured by ELISA of IL-2 and IFN-γ produced at 24 h supernatants. Similar results were seen in 3 independent experiments. *p<0.05

FIG. 4B is a graphs showing expression of class II MHC molecules in CIITA^{null} EC was rescued upon reintroduction of functional copy of CIITA by retroviral transduction.

FIG. 4C is a panel of plots showing CIITA^{null} EC lost the ability to activate alloreactive CD4+ memory T lymphocytes as measured by CFSE dilution and HLA-DR activation marker, which was rescued with CIITA retrovirus. Representative plots and mean±SD from n=4 replicates. Similar results were seen in 3 independent experiments. *indicates statistically significant difference by post hoc Bonferroni correction.

FIG. 5A is a panel of graphs showing doxycycline-induced mutagenesis and analysis and recovery of CIITA^{null} EC. EC were transduced with tretracycline-inducible Cas9 vector to express high levels of Cas9 in the presence of doxycycline and remain viable. Representative plots and mean±SD of triplicate samples from 3 independent experiments. *p<0.05

FIG. 5B is a graph showing the comparison of three CIITA-specific sgRNA on inhibition of IFN-γ-induced expression of MHC class II in absence or presence of doxycycline. The data demonstrated reproducible Cas9-mediated ablation of CIITA that was minimal in the absence of doxycycline. Representative plots and mean±SD of triplicate samples from 3 independent experiments. *p<0.05

FIG. 5C is a graph showing qRT-PCR analysis of FACS isolated WT and HLA-DR^{neg} EC for CIITA, HLA-DRA, and CXCL10 transcripts. Representative plots and mean±SD of triplicate samples from 3 independent experiments. *p<0.05

FIG. 5D is a graph showing that transient culture with ROCK-selective inhibitor Y-27632 enhanced recovery of FACS isolated CIITA^{null} EC (n=4 independent isolations).

FIG. 6A in an illustration showing CD58-specific sgRNA ablated surface expression of CD58 on EC whereas EC transduced with CIITA-specific sgRNA expressed levels equivalent to unmodified EC.

FIG. 6B is a graph showing the high efficiency disruption of CD58 by CRISPR/Cas9. CIITA and CD58 sgRNA were multiplexed together to generate CD58^{neg}HLA-DRA^{neg}CD31⁺ cells. Results are representative from 2 independent experiments using distinct donors.

FIG. 7A is an image showing CIITA^{null} EC formed blood conduits in vivo. All perfused conduits formed from WT and CIITA^{null} EC were positive for human CD31. Representative images of vascularized implants from 9 mice.

FIG. 7B is an image showing significant investment in some conduits by murine SM α-actin positive mesenchymal cells. Representative images of vascularized implants from 9 mice.

FIGS. 8A-8B are a series of graphs and images illustrating the finding that HLA-DR blockade reduced acute T cell-mediated injury to implanted allogeneic vessel segments in vivo. In an MHC-mismatched model of arterial rejection by allogeneic T cells (n=6 per group), blockade of class II MHC by anti-HLA-DRα F(ab)′2 fragment reduced intimal area expansion and increased lumen area as measured by H&E and EVG staining (scale bar: 50 μm) (FIG. 8A) and reduced disruption of EC lining the vessel lumen, a hallmark of intimal arteritis/endothelialitis, as measured by percent of circumferential coverage (scale bar: 20 μm) (FIG. 8B), both at 21 days. *P<0.05, 2-tailed Student's t test.

FIGS. 9A-9B are a series of images and graphs showing HLA-DR blockade reduced CD8⁺ T cell infiltration into and CTL development within implanted allogeneic vessel segments in vivo. Anti-HLA-DRα F(ab)′2 fragment significantly (n=6 per group) reduced total intimal T cell infiltration as detected by immunofluorescence (scale bar: 20 μm) (FIG. 9A) and CD8+T cell-associated transcripts of cytokines and CTL effector molecules in rejected artery grafts at 21 days (FIG. 9B) as detected by qPCR normalized to either total mRNA or T cell-specific mRNA as indicated on the y axis. *P<0.05, 2-tailed Student's t test.

FIGS. 10A-10B are a series of graphs showing anti-CIITA siRNA selectively inhibited induction of CIITA and class II MHC molecules in human EC. Cultured human EC were transfected with anti-CIITA siRNA or control siRNA on day 0, treated with IFN-γ at 24 hours, and analyzed on day 3. Specific transcripts were measured by qPCR (FIG. 10A), and protein was assessed by FACS (FIG. 10B). Similar results were seen in four independent experiments. ***P<0.0005, 2-tailed Student's t test.

FIGS. 11A-11B are a series of graphs showing siRNA-mediated knockdown of CIITA in EC directly inhibited CD4⁺ but not CD8+ T_{EM} alloresponses. Anti-CIITA siRNA inhibited activation of CD4⁻ T_{EM} (10:1 T cell/EC ratio) (FIG. 11A) but not activation of CD8⁺ T_{EM} (30:1 T cell/EC ratio) (FIG. 11B), as measured by ELISA at 24 hours for IL-2 and IFN-γ and proliferation by CFSE dilution and flow cytometric analysis at 7 days. Note that the majority of CD8⁺ T_{EM} have divided only once, whereas CD4⁺ T_{EM} underwent multiple rounds of proliferation. Similar results were seen in three independent experiments. *P<0.05, 2-tailed Student's t test.

FIGS. 12A-12C are a series of graphs showing activation of CD4⁺ T_{EM} by allogeneic EC was necessary to enhance CD8⁺ T_{EM} responses to the same EC. Addition of CD4⁺ T_{EM} to EC/CD8+T_{EM} cocultures enhanced CD8⁺ T_{EM} expansion as measured by CFSE dilution and flow cytometry at 7 days (FIG. 12A), but CD4⁺ T_{EM} did not enhance survival of CD8⁺ T_{EM} (FIG. 12B) as measured by viability. (FIG. 12C) Knockdown of CIITA expression by siRNA, to prevent CD4⁺ T_{EM} activation, inhibited CD4⁺ T_{EM} enhancement of CD8⁻ T_{EM} expansion at 7 days as measured by CFSE dilution and flow cytometric analysis at 7 days. Note that the inclusion of activated CD4⁺ T_{EM} increaseed the rounds of replication of CD8⁺ T_{EM} (inset boxes). Similar results were seen in three independent experiments. *P<0.05, 2-tailed Student's t test.

FIGS. 13A-13E are a series of graphs showing CD4+ T_{EM} enhancement of CD8+T_{EM} responses were mediated by secreted IL-2. (FIG. 13A) EC precultured with CD4+ T_{EM} and subsequently isolated by FACS had not increased their capacity to activate CD8+T_{EM}. (FIG. 13B) When separated by a semipermeable transwell membrane, CD4+ T_{EM} enhanced CD8+T_{EM} alloresponses. (FIG. 13C) CM generated by EC-activated CD4+ T_{EM} was sufficient to enhance proliferation of CD8+T_{EM}. Immunoabsorption of IL-2 removed that capacity, whereas addition of IL-2 to control CM mimics activated CM. (FIG. 13D) Similarly, CM induced phosphorylates STATS in CD25+ but not CD25-CD8+ T_{EM} in an IL-2-dependent manner. (FIG. 13E) In addition, CM enhanced expression of perforin and promoted CTL killing in an IL-2-dependent manner. Similar results were seen in three independent experiments. *P<0.05, by 2-tailed Student's t test (FIG. 13B) or by 1-way ANOVA with Bonferroni post-hoc test (FIG. 13A and 13C-13E).

FIG. 14 is a series of images and a graph showing genetic ablation of CIITA in EC by CRISPR/Cas9 reduced T cell-mediated destruction of synthetic vessels in vivo. Synthetic human microvessels formed from WT or CIITA^{null} EC were implanted s.c. in the abdominal wall of C.B-17 SCID/bg mice. After 2 weeks, mice were adoptively transferred with peripheral blood mononuclear cells (PBMC). In the absence of circulating T cells, WT and CIITA^{null} EC formed equivalent numbers of vessels (n=3 each). In the presence of circulating T cells, however, there was a significantly higher loss of perfused vessels formed from WT compared with CIITA^{null} EC (n=3 each). Scale bar: 100 μm. *P<0.05, 2-tailed Student's t test.

FIGS. 15A-15B are a set of graphs showing F(ab)′2 fragments of anti-HLA-DRα mAb did not affect circulating human CD3⁺ cells in vivo but effectively reduced recognition of EC by CD4⁺ T_{EM} in vitro. FIG. 15A shows anti-HLA-DRα F(ab)′2 fragment did not affect adoptive transfer of human CD3⁺ cells to the peripheral circulation of C.B-17 SCID/bg mice (n=3). FIG. 15B shows the F(ab)′2 fragments retained blocking activity equivalent to the full length IgG as measured by CD4⁺ T_{EM} proliferation to allogeneic EC (n=3). Similar results were seen in three independent experiments. * significant by one-way ANOVA with Bonferroni post-hoc test.

FIG. 16 is a series of plots showing magnetic bead immunoselection of CD4⁺ and CD8⁺ T_{EM} from human PBMC resulted in >95% purity for cells that are CD62L⁻CCR7⁻ CD45RO⁺.

FIG. 17 is a series of graphs showing addition of CD4⁺ T_{EM} to EC/CD8⁺ T_{EM} co-cultures did not significantly increase expression of CD25, a marker of TCR engagement at 48 h detected by flow cytometry. (n=3) Similar results were seen in two independent experiments. *p<0.05, 2-tailed Student's t test.

FIG. 18 is a series of plots showing concomitant activation of CD4⁺ T_{EM} increased frequency of perforin⁺CD8⁺ T cells in alloreactive population)(CFSE^{lo}) but not non-reactive (CFSE^{hi}). CD8⁺ T_{EM} alone or CD4⁺ and CD8⁺ T_{EM} were cultured with EC that had been pre-treated with anti-CIITA or control siRNA and subsequently activated with IFN-γ, 48 h prior. At 7 d, cells were harvested and analyzed for CD8⁺ expression, CFSE dilution and intracellular perforin expression by flow cytometry (n=4). Similar results were seen in three independent experiments. *significant by one-way ANOVA with Bonferroni post-hoc test.

FIGS. 19A-19B are a series of plots showing genetic ablation of CIITA in human EC derived from endothelial colony-forming cells (HECFC)-derived EC by CRISPR/Cas9 prevented CD4⁺ T_{EM} from enhancing CD8⁺ T_{EM} alloresponses. FIG. 19A shows genetic ablation of CIITA in EC eliminated expression of class II MHC molecules as measured by qRT-PCR. FIG. 19B shows CD8⁺ T_{EM} or CD8⁺/CD4⁺ T_{EM} were co-cultured with unmodified or CIITA^{null} EC pre-treated with IFN-γ to re-induce CIITA and measured by CFSE dilution and flow cytometric analysis at 7 d. Similar results were seen in two independent experiments with n=3 donors. ***p<0.0005, 2-tailed Student's t test.

FIGS. 20A-20B are a set of images and plots showing depletion of CD8⁻ cells from PBMC inoculum prior to adoptive transfer resulted in absence of CD8⁺ T cells in the peripheral blood and reduced the extent of allogeneic human EC microvessel destruction. FIG. 20A shows analysis of circulating human CD3⁺ subsets from blood taken 10 d after adoptive transfer of 2×10⁸ human PBMC. As quantified by flow cytometry, pre-depletion of CD8⁺ effectively prevented appearance of CD8⁻ cells in the peripheral circulation (n=3). FIG. 20B shows adoptive transfer of whole PBMC into animals bearing synthetic human microvessels resulted in loss of perfused vessels at 10 d, whereas removal of CD8⁺ from the PBMC inoculum significantly inhibited vessel loss (n=3). Similar results were seen in two independent experiments. *p<0.05, 2-tailed Student's t test.

Abbreviations: EVG—Elastia-van Gieson; MHC—major histocompatibility complex; EC—endothelial cells; CTL—cytotoxic T cell; CIITA—class II MHC transactivator; T_{EM}—effector memory T cell; FVD—fixable viability dye; CM—conditioned medium; bg—beige; PBMC, peripheral blood mononuclear cells.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a cell that has been sufficiently stimulated to induce detectable cellular activation. Activation can be associated with induced proliferation, cytokine production, and detectable effector functions. The term “activated cell” refers to, among other things, cells that are undergoing cell division.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia areata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cleavage” refers to the breakage of covalent bonds, such as in the backbone of a nucleic acid molecule. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

The term “CRISPR/CAS,” “clustered regularly interspaced short palindromic repeats system,” or “CRISPR” refers to DNA loci containing short repetitions of base sequences. Each repetition is followed by short segments of spacer DNA from previous exposures to a virus. Bacteria and archaea have evolved adaptive immune defenses termed CRISPR-CRISPR-associated (Cas) systems that use short RNA to direct degradation of foreign nucleic acids. In bacteria, the CRISPR system provides acquired immunity against invading foreign DNA via. RNA-guided DNA cleavage.

In the type II CRISPR/Cas system, short segments of foreign DNA, termed “spacers” are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Recent work has shown that target recognition by the Cas9 protein requires a “seed” sequence within the crRNA and a conserved dinucleotide-containing protospacer adjacent motif (PAM) sequence upstream of the crRNA-binding region.

To direct Cas9 to cleave sequences of interest, crRNA-tracrRNA fusion transcripts, hereafter referred to as “guide RNAs” or “gRNAs” may be designed, from human U6 polymerase III promoter. CRISPR/CAS mediated genome editing and regulation, highlighted its transformative potential for basic science, cellular engineering and therapeutics.

The term “CRISPRi” refers to a CRISPR system for sequence specific gene repression or inhibition of gene expression, such as at the transcriptional level.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to a decrease or ablation of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expand” as used herein refers to increasing in number, as in an increase in the number of cells. In one embodiment, the cells that are expanded ex vivo increase in number relative to the number originally present in the culture. In another embodiment, the cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. As applied to the nucleic acid or protein, “homologous” as used herein refers to a sequence that has about 50% sequence identity. More preferably, the homologous sequence has about 75% sequence identity, even more preferably, has at least about 80%, 85%, 90%, 91%, 92%, 93%, 94%, 95%, 96%, 97%, 98%, 99% sequence identity.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “immune response” as used herein is defined as a cellular response to an antigen that occurs when lymphocytes identify antigenic molecules as foreign and induce the formation of antibodies and/or activate lymphocytes to remove the antigen.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The term “guide nucleic acid sequence,” relates to a polynucleotide sequence that can form a complex with a Cas endonuclease and enables the Cas endonuclease to recognize and optionally cleave a DNA target site. The guide nucleic acid sequence can be a single guide nucleic acid sequence or a double guide nucleic acid sequence. The guide nucleic acid sequence sequence can be a RNA sequence, a DNA sequence, or a combination thereof (a RNA-DNA combination sequence). Optionally, the guide nucleic acid sequence can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, Phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. In some embodiment of this disclosure, the guide nucleic acid sequence does not solely comprise ribonucleic acids (RNAs). A guide nucleic acid sequence that solely comprises ribonucleic acids is also referred to as a “single guide RNA.”

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

The guide nucleic acid sequence may be complementary to one strand (nucleotide sequence) of a double stranded DNA target site. The percentage of complementation between the guide nucleic acid sequence and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The guide nucleic acid sequence can be at least 10, 11, 12, 13, 14, 15, 16, 17, 18, 19, 20, 21, 22, 23, 24, 25, 26, 27, 28, 29, 30, 31, 32, 33, 34, 35 or more nucleotides in length. In some embodiments, the guide nucleic acid sequence comprises a contiguous stretch of 10 to 40 nucleotides. The variable targeting domain can be composed of a DNA sequence, a RNA sequence, a modified DNA sequence, a modified RNA sequence (see for example modifications described herein), or any combination thereof.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By the term “specifically binds,” as used herein with respect to a polynucleotide is meant specifically hybridizes to another polynucleotide, with respect to a polypeptide is meant recognition of a specific binding partner, but does not substantially hybridize or bind other molecules in a sample. For example, a polynucleotide with sufficient complementary to another nucleotide sequence may specifically bind to that sequence and sequences with a similar level of complementarity. The complementary sequence may also include different allelic forms of the sequence. In another example, an antibody that specifically binds to an antigen from one species may also bind to that antigen from one or more species. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

As used herein, “substantially purified” refers to a cell or molecule that is essentially free of other materials, such as cellular components. A substantially purified cell also refers to a cell which has been separated from other cell types with which it is normally associated in its naturally occurring state. In some instances, a substantially purified cell or molecule refers to a homogenous population of cells or homogenous composition of the molecule. In other instances, this term refers to a cell or molecule that has been separated from components they are naturally associated with in the natural state.

A “target site” or “target sequence” refers to a genomic nucleic acid sequence that defines a portion of a nucleic acid to which a binding molecule may specifically bind under conditions sufficient for binding to occur.

The terms “target locus,” “genomic target site,” “genomic target sequence,” and “genomic target locus” are used interchangeably herein and refer to a nucleic acid sequence in the genome (including mitochondrial DNA) of a cell at which a double-strand break is induced in the cell genome by a Cas endonuclease. The target site can be an endogenous site in the genome of an cell or organism, or alternatively, the target site can be heterologous to the cell or organism and thereby not be naturally occurring in the genome, or the target site can be found in a heterologous genomic location compared to where it occurs in nature.

As used herein, terms “endogenous target sequence” and “native target sequence” are used interchangeable herein to refer to a target sequence that is endogenous or native to the genome of a cell or organism and is at the endogenous or native position of that target sequence in the genome of a cell or organism. Cells include, but are not limited to animal, bacterial, fungal, insect, yeast, and plant cells as well as plants and seeds produced by the methods described herein.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention includes methods and compositions for modifying endothelial cells for improved vascular self-assembly. In some embodiments, the invention includes a modified endothelial cell and methods of generating and using the modified endothelial cell in a pharmaceutical composition. Other embodiments include methods of promoting vascular self-assembly and regenerating vascular structures in a subject in need thereof. In particular, the modified endothelial cell has reduced immunogenicity in an allogeneic environment in the subject, while still capable of forming vascular structures.

### Endothelial Cells

The present invention includes a modified endothelial cell comprising a nucleic acid capable of downregulating gene expression of a gene selected from the group consisting of a class I and class II major histocompatibility complex (MHC) molecule, CD58, CIITA, NRLC5, beta2 microglobulin, ICOS-ligand, 4-1BB ligand, O×40 ligand, GITR ligand, interleukin 1 alpha, interleukin 6, ICAM-1, ICAM-2, VCAM-1, E-selectin, P-selectin and combinations thereof, and wherein the modified endothelial cell has reduced immunogenicity in an allogeneic environment. In one aspect, the invention includes a modified endothelial cell comprising a Cas expression vector and a guide nucleic acid sequence specific for a gene, wherein Cas is expressed and interacts with the guide nucleic acid sequence to mutate a loci for the gene, and wherein the modified endothelial cell has reduced immunogenicity in an allogeneic environment. In one aspect, the invention includes a modified endothelial cell comprising an inducible Cas9 expression vector; a single guide RNA specific for CD58; and a single guide RNA specific for CIITA, wherein induction of Cas9 expression results in the cell having reduced immunogenicity in an allogeneic environment.

To produce stable genetic alterations of differentiated human EC, three different technical advances were combined. First, untransformed human EC cultures were produced from outgrowth of endothelial colony forming cells (ECFC), also known as late outgrowth EC or endothelial progenitor cells, isolated from cord blood (Ingram D A, et al. Blood. 2004; 104:2752-2760). Second, lentiviral vectors were used to introduce tetracycline-inducible Cas9 and constitutively expressed RNA guide strands (Wang T, et al. Science. 2014; 343:80-84). Third, cloning conditions were optimized to routinely produce multiple different colonies with distinct bi-allelic deletions (Watanabe K, et al. Nature biotechnology. 2007; 25:681-686). The combination of these advances allows highly efficient and simple gene disruption in human EC. As proof-of-principle, EC lacking the transcriptional activator CIITA, the master regulator of MHC II expression, was generated and the cells were found to lose their ability to express class II MHC molecules, thereby eliminating their ability to activate allogeneic CD4+ T cells without altering their basic properties, including the capacity to self-assemble into vascular structures in vivo.

### Downregulation of Gene Expression

The present invention includes a modified EC comprising a nucleic acid capable of downregulating gene expression in the EC. The gene expression may be downregulated, knocked-down, decreased, and/or inhibited by, for example a siRNA, a CRISPR system, and other methods known in the art.

In one embodiment, the nucleic acid capable of downregulating gene expression includes a siRNA and a CRISPR system. Application of antisense oligonucleotides or RNAi often results in partial knockdown and, in the case of siRNA, for a limited duration. In embodiments that comprise a siRNA, the siRNA may, according to the present invention, target a class I and class II major histocompatibility complex (MHC) molecule, CD58, CIITA, NRLC5, beta2 microglobulin, ICOS-ligand, 4-1BB ligand, O×40 ligand, GITR ligand, interleukin 1 alpha, interleukin 6, ICAM-1, ICAM-2, VCAM-1, E-selectin, P-selectin and combinations thereof. In one such embodiment, the siRNA comprises SEQ ID NO:7.

Unfortunately, human ECs are typically hard to transfect, have a limited replicative lifespan and are not amenable to cloning after stable genetic manipulation. Permanent gene disruption by CRISPR/Cas9 is a transformative technology that utilizes the RNA-guided Cas9 endonuclease to introduce DNA double stranded breaks which trigger error-prone repair pathways that can result in frame shift mutations.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In embodiments that comprise a CRISPR system, the CRISPR system further comprises a Cas expression vector and a guide nucleic acid sequence specific for the gene. In some embodiments, modified endothelial cell are generated by introducing a Cas expression vector into the ECFCs and a guide nucleic acid sequence specific for a gene a Cas expression vector into endothelial colony forming cells (ECFCs) and a guide nucleic acid sequence specific for a gene. In one embodiment, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combination thereof.

In one embodiment, the Cas expression vector comprises a viral Cas expression vector, such as a Sendai viral vector, an adenoviral vector, an adeno-associated viral vector, a retroviral vector, or a lentiviral vector.

In another embodiment, the Cas expression vector is under the control of an inducible promoter. The Cas expression vector may include an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector. In some embodiments, the Cas expression vector transposon includes an inducible promoter that is inducible by the addition of an antibiotic but does not require antibiotic resistance. In such an embodiment, the Cas expression vector is under the control of an inducible promoter. In one embodiment, inducing the Cas expression vector comprises exposing the ECFCs to an agent that activates an inducible promoter in the Cas expression vector.

The CRISPR system also includes a guide nucleic acid sequence. The guide nucleic acid sequence includes a RNA sequence, a DNA sequence, or a combination thereof (a RNA-DNA combination sequence). The guide nucleic acid sequence can be a single molecule or a double molecule. In one embodiment, the guide nucleic acid sequence comprises a single guide RNA.

In one embodiment, the single guide RNA comprises at least one sequence selected from the group consisting of SEQ ID NO: 1, SEQ ID NO: 2, SEQ ID NO: 3, and SEQ ID NO: 4.

The guide nucleic acid sequence targets a specific gene. The sequence that the guide nucleic acid sequence targets may be within a loci of the gene and cleaved by a Cas endonuclease. In one embodiment, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Alternatively, the guide nucleic acid sequence may target any gene. In one embodiment, the gene may include a class I and class II major histocompatibility complex (MHC) molecule, CD58, CIITA, NRLC5, beta2 microglobulin, ICOS-ligand, 4-1BB ligand, O×40 ligand, GITR ligand, interleukin 1 alpha, interleukin 6, ICAM-1, ICAM-2, VCAM-1, E-selectin, P-selectin and combinations thereof. In one aspect, the invention includes a modified endothelial cell comprising an inducible Cas9 expression vector; a single guide RNA specific for CD58; and a single guide RNA specific for CIITA, wherein induction of Cas9 expression results in the cell having reduced immunogenicity in an allogeneic environment.

### Introduction of Nucleic Acids

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. The nucleic acids can be introduced into target cells using commercially available methods, which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

In one embodiment, introducing the Cas expression vector comprises transducing the ECFCs with a viral Cas expression vector. In one such embodiment, the viral Cas expression vector may be a Sendai viral vector, an adenoviral vector, an adeno-associated viral vector, a retroviral vector, or a lentiviral vector.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Culturing Endothelial Cells

The present invention includes methods for generating a modified endothelial cell (EC) by culturing endothelial colony forming cells (ECFCs) and introducing into the cell a nucleic acid capable of downregulating gene expression of an endogenous gene into the cell. In one aspect, the invention includes a modified EC generated by culturing endothelial colony forming cells (ECFCs) and introducing a nucleic acid capable of downregulating gene expression of an endogenous gene selected from the group consisting of a class I and class II major histocompatibility complex (MEW) molecule, CD58, CIITA, NRLC5, beta2 microglobulin, ICOS-ligand, 4-1BB ligand, O×40 ligand, GITR ligand, interleukin 1 alpha, interleukin 6, ICAM-1, ICAM-2, VCAM-1, E-selectin, P-selectin and combinations thereof in the ECFCs, wherein the modified EC has reduced immunogenicity in an allogeneic environment.

In some embodiments, introducing the nucleic acid further comprises introducing a nucleic acid selected from the group consisting of a siRNA and a CRISPR system. In one such embodiment, the siRNA comprises SEQ ID NO:7. In another such embodiment, the CRISPR system comprises a Cas expression vector and a guide nucleic acid sequence specific for a gene. The Cas expression vector and the guide nucleic acid are introduced into the ECFCs and Cas expression is induced from the Cas expression vector such that the expressed Cas interacts with the guide nucleic acid sequence to mutate a loci for the gene.

In one embodiment, introducing the Cas expression vector comprises transducing the ECFCs with a viral Cas expression vector, such as a Sendai viral vector, an adenoviral vector, an adeno-associated viral vector, a retroviral vector, and a lentiviral vector. In another embodiment, introducing the Cas expression vector comprises exposing the ECFCs to an agent that activates an inducible promoter, as described elsewhere herein, in the Cas expression vector.

Prior to modification, ECs are obtained from a subject. Non-limiting examples of subjects include humans, dogs, cats, mice, rats, and transgenic species thereof. Preferably, the subject is a human. ECs can be obtained from a number of sources, including peripheral blood mononuclear cells, bone marrow, lymph node tissue, spleen tissue, umbilical cord, and tumors. In certain embodiments, ECs can be obtained from a unit of blood collected from a subject using any number of techniques known to the skilled artisan, such as Ficoll separation. In certain embodiments, any number of EC lines, preferably endothelial progenitor cell lines or stem cell lines that may be differentiated into endothelial progenitor cells, are available in the art and may be used. In one embodiment, endothelial progenitor cells from the circulating blood, bone marrow or umbilical cord blood of an individual. The endothelial progenitor cells collected may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, endothelial progenitor cells are isolated from peripheral blood, bone marrow or umbilical cord blood by lysing the red blood cells and depleting the monocytes, for example, by centrifugation through a PERCOLL™ gradient. In any event, a specific subpopulation of endothelial progenitor cells can be further isolated by positive or negative selection techniques.

The cord blood mononuclear cells so isolated can be depleted of cells expressing certain antigens, including, but not limited to, CD4, CD8, CD14, CD19 and CD56. Depletion of these cells can be accomplished using an isolated antibody, a biological sample comprising an antibody, such as ascites, an antibody bound to a physical support, and a cell bound antibody.

Enrichment of an EC population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD31+CD34+CD45+cells by negative selection, a monoclonal antibody cocktail typically includes antibodies to CD3, CD4, CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

ECs can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° per minute and stored in the vapor phase of a liquid nitrogen storage tank. Other methods of controlled freezing may be used as well as uncontrolled freezing immediately at -20° C. or in liquid nitrogen.

In one embodiment, the population of ECs is comprised within cells such as peripheral blood mononuclear cells, cord blood cells, a purified population of ECs, and an EC line.

In one embodiment, the ECs, such as endothelial colony forming cells (ECFCs), are obtained from cord blood endothelial progenitor cells. In another embodiment, the endothelial cells, ECFCs, are differentiated from cord blood mononuclear cells. In yet another embodiment, the endothelial cells, ECFCs, are differentiated by culturing the endothelial progenitor cells with extracellular matrix proteins, such as gelatin coated tissue cultures dishes. The cultures may further be supplemented with serum, cytokines such as vascular endothelial growth factor (VEGF), bovine brain extract, and other components known in the art.

Following culturing, the ECs can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The EC medium may be replaced during the culture of the ECs at any time. Preferably, the EC medium is replaced about every 2 to 3 days. The ECs are then harvested from the culture apparatus whereupon the ECs can be used immediately or cryopreserved to be stored for use at a later time. In one embodiment, the invention includes cryopreserving the cultured ECs. The cryopreserved ECs are thawed prior to modification.

In one aspect, the method of culturing the ECs can further comprise introducing the Cas expression vector into the ECs. The ECs may then be cryopreserved prior to induction of the expression of Cas. In yet another embodiment, the cryopreserved ECs are thawed and then Cas expression is induced.

The culturing step as described herein (contact with agents as described herein) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), insulin, VEGF, GM-CSF, IL-2, IL-10, IL-12, IL-15, TGF-beta, and TNF-α. or any other additives for the growth of cells known to the skilled artisan. Other additives for the growth of cells include, but are not limited to, surfactant, plasmanate, and reducing agents such as N-acetyl-cysteine and 2-mercaptoethanol. Media can include EGM, RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

### Therapy

The modified ECs described herein may be included in a composition for therapy. The composition may include a pharmaceutical composition and further include a pharmaceutically acceptable carrier. A therapeutically effective amount of the pharmaceutical composition comprising the modified ECs may be administered.

In one aspect, the invention includes a method for promoting vascular self-assembly in a subject in need thereof. The method comprises administering to a subject an effective amount of a modified EC capable of forming vascular structures. The modified EC comprises a nucleic acid capable of downregulating gene expression of an endogenous gene selected from the group consisting of a class I and class II major histocompatibility complex (MEW) molecule, CD58, CIITA, NRLC5, beta2 microglobulin, ICOS-ligand, 4-1BB ligand, O×40 ligand, GITR ligand, interleukin 1 alpha, interleukin 6, ICAM-1, ICAM-2, VCAM-1, E-selectin, P-selectin and combinations thereof. The modified EC has reduced immunogenicity in an allogeneic environment while maintaining the capacity to form vascular structures.

In another aspect, the invention includes a method for regenerating vascular structures in a subject in need thereof. The method comprises administering to a subject an effective amount of a modified EC capable of forming vascular structures. The modified EC comprises a nucleic acid capable of downregulating gene expression of an endogenous gene selected from the group consisting of a class I and class II major histocompatibility complex (MEW) molecule, CD58, CIITA, NRLC5, beta2 microglobulin, ICOS-ligand, 4-1BB ligand, O×40 ligand, GITR ligand, interleukin 1 alpha, interleukin 6, ICAM-1, ICAM-2, VCAM-1, E-selectin, P-selectin and combinations thereof. The modified EC has reduced immunogenicity in an allogeneic environment while maintaining the capacity to form vascular structures.

The modified ECs generated as described herein are uniform and possess endothelial cell function. Further, the ECs can be administered to an animal, preferably a mammal, even more preferably a human, to aid in vascular self-assembly while having reduced immunogenicity, such as enhancing allograft tolerance, and the like. In addition, the cells of the present invention can be used for the treatment of any condition in which diminished vascularity or tissues lacking vascularity, especially in regeneration of new tissues, is present. In one aspect, the invention includes promoting regeneration of a tissue in a subject in need thereof comprising administering to the subject a therapeutically effective amount of a pharmaceutical composition comprising a population of modified ECs.

In another embodiment, the ECs described herein may be used for the manufacture of a medicament for regenerating vascular structures in a subject in need thereof In yet another embodiment, the ECs described herein may be used in the manufacture of an engineered tissue or organ.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

The cells of the invention to be administered may be autologous, allogeneic or xenogeneic with respect to the subject undergoing therapy.

The administration of the cells of the invention may be carried out in any convenient manner known to those of skill in the art. The cells of the present invention may be administered to a subject by aerosol inhalation, injection, ingestion, transfusion, implantation or transplantation. The cells described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

In one embodiment, the ECs described herein are incorporated into an engineered tissue or organ. The cells described herein can be incorporated into any number of matrices as part of the tissue engineered organ. The present invention utilizes such matrices within the novel context of acting as an artificial organ to support, maintain, or modulate a system. Accordingly, the present invention can utilize those matrix compositions and formulations which have demonstrated utility in tissue engineering. Accordingly, the type of matrix that may be used in the compositions, devices and methods of the invention is virtually limitless and may include both biological and synthetic matrices. In one particular example, the compositions and devices set forth by U.S. Pat. Nos. 5,980,889; 5,913,998; 5,902,745; 5,843,069; 5,787,900; or 5,626,561 are utilized, as such these patents are incorporated herein by reference in their entirety. Matrices comprise features commonly associated with being biocompatible when administered to a mammalian host. Matrices may be formed from natural and/or synthetic materials. The matrices may be non-biodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release of seeded cells or produced cytokine or other active agent. In certain embodiments, the matrix of the present invention is flexible and elastic, and may be described as a semisolid scaffold that is permeable to substances such as inorganic salts, aqueous fluids and dissolved gaseous agents including oxygen.

A matrix is used herein as an example of a biocompatible substance. However, the current invention is not limited to matrices and thus, wherever the term matrix or matrices appears these terms should be read to include devices and other substances which allow for cellular retention or cellular traversal, are biocompatible, and are capable of allowing traversal of macromolecules either directly through the substance such that the substance itself is a semi-permeable membrane or used in conjunction with a particular semi-permeable substance.

### Pharmaceutical Compositions

Pharmaceutical compositions of the present invention may comprise a modified EC population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an effective amount”, “an anti-immune response effective amount”, “an immune response-inhibiting effective amount”, or “therapeutic amount” is indicated, the precise amount of the compositions of the present invention to be administered can be determined by a physician with consideration of individual differences in age, weight, immune response, and condition of the patient (subject). It can generally be stated that a pharmaceutical composition comprising the modified ECs described herein may be administered at a dosage of 10⁴ to 10⁹ cells/kg body weight, preferably 10⁵ to 10⁶ cells/kg body weight, including all integer values within those ranges. Modified EC compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to draw blood (or have an apheresis performed), modify ECs therefrom according to the present invention, and reinfuse the patient with these modified ECs. This process can be carried out multiple times every few weeks. In certain embodiments, modified ECs can be obtained from blood draws of from 10 ml to 400 ml. In certain embodiments, ECs are obtained from blood draws of 20 ml, 30 ml, 40 ml, 50 ml, 60 ml, 70 ml, 80 ml, 90 ml, or 100 ml.

In certain embodiments of the present invention, ECs may be expanded and modified using the methods described herein, or other methods known in the art where ECs are expanded to therapeutic levels and administered to a patient in conjunction with (e.g., before, simultaneously or following) any number of relevant treatment modalities.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

The materials and methods employed in Example 1 experiments are now described.

Isolation of ECFC. Umbilical cord blood was obtained with informed consent under a protocol approved by the Yale Human Investigation Committee. ECFC were differentiated from cord blood mononuclear cells in vitro as “late outgrowth” cells, as previously described (Shepherd B R, et al. The FASEB journal. 2006; 20:1739-1741), and then expanded in culture. Cultures were serially propagated in gelatin (Sigma-Aldrich, St. Louis, Mo.) coated tissue culture flasks with EGM-2/5% FBS (Lonza, Walkersville, Md.). After transduction with tetracycline-inducible Cas9 lentiviral vector, cultures were maintained under the same medium using tetracycline-free FBS (Clontech, Mountain View, Calif.).

Isolation of memory T lymphocytes. PBMCs were collected with informed consent by leukapheresis from anonymized healthy volunteer donors under a protocol approved by the Yale Human Investigation Committee. Mononuclear cells were further enriched by density gradient centrifugation of leukapheresis products using Lymphocyte Separation Medium (MP Biomedicals, Santa Ana, Calif.) according to the manufacturer's protocol. Purified PBMCs were cryopreserved in 10% DMSO-90% FBS in liquid nitrogen before use. CD4+ T cells were isolated from PBMCs using Dynabeads CD4+ Positive Isolation Kit (Invitrogen/Life Technologies, Carlsbad, Calif.) per manufacturer's protocol. Naïve and activated T cells as well as monocytes were removed by negative selection using anti-CD45RA (Clone: H100, eBioscience, San Diego, Calif.) and anti-HLA-DR mAb (Clone: LB3.1, gift of J. Strominger, Harvard University, Cambridge, Mass.) at a concentration of 5 μg/ml for 20 min, washed twice, and magnetically depleted using pan-mouse IgG beads (Invitrogen). The population obtained by this procedure was routinely >98% HLA-DR⁻CD45RA⁻CD⁺ T lymphocytes by flow cytometry.

Lentiviral vector construction. Guide RNA sites in CIITA exonic loci were identified using the online optimized design software at http://crispr.mit.edu. The highest scoring sgRNA, which had no off-target sequences with perfect matches in the human genome and the nearest coding off-target sites contained +3 mismatches, were used to create IDT gBlock segments composed of Xhol restriction site, U6 promoter, sgRNA target sequence, chimeric sgRNA scaffold, and Nhel restriction site (Integrated DNA Technologies, Coralville, Iowa). gBlocks were cloned into pLX-sgRNA (Wang T, et al. Science. 2014; 343:80-84) (Addgene plasmid #50662), which contained the blasticidin resistance gene. The CIITA sgRNA targeting sequences used in this study include: GCTGAACTGGTCGCAGTTGA (SEQ ID NO: 1), GATATTGGCATAAGCCTCCC (SEQ ID NO: 2), GTCAACTGCGACCAGTTCAGC (SEQ ID NO: 3). The CD58 targeting sequence: TGGTTGCTGGGAGCGACGC (SEQ ID NO: 4), which was prepended with a G nucleotide for efficient U6 transcription. A separate lentiviral vector encoding tetracycline-inducible FLAG-Cas9 (pCW-Cas9, Addgene plasmid #50661) and puromycin resistance gene was used to create TetOn-Cas9-EC.

Lentiviral packaging and transduction. Lentiviral vector plasmids were co-transfected with psPAX2 (Addgene plasmid #12260) and CMV VSV-G (Addgene plasmid #8454) packaging plasmids into human 293T (ATCC, Manassas, Va.) cells using Lipofectamine 2000 (Invitrogen) per manufacturer's protocol. Lentiviral supernatant was collected at 48 h and 72 h, filtered through 0.45 uM filter, and used to transduce EC in C-12 well plates in the presence of 8 ug/ml polybrene (Sigma-Aldrich). TetOn-Cas9-EC were generated by transduction with pCW-Cas9 vector for 8 h and after 24 h, drug-selected with 1 μg/ml puromycin (Invitrogen) for 3 d. Cas9 expression was confirmed by intracellular flow cytometric analysis using APC-conjugated anti-FLAG mAb (Clone: L5, BioLegend, San Diego, Calif.) after doxycycline treatment. TetOn-Cas9-EC were then treated with lentiviral supernatant containing CIITA or CD58 specific sgRNA vectors and 8 μg/ml polybrene for 8 h. After 24 h, cells were selected with 10 μg/ml blasticidin (Invitrogen) and 1 μg/ml doxycycline (Sigma-Aldrich) for 5 d. When multiplexed, a 50/50 mixture of supernatants containing CIITA and CD58 sgRNA lentiviral vectors was used.

FACS Analysis and Isolation of CRISPR/Cas9-modified EC. After transduction and selection, EC were stimulated with IFN-γ (50 ng/ml, Invitrogen) to up regulate MHC II. EC were harvested with trypsin (TrypLE Express, Invitrogen) and subsequently stained with directly conjugated Pacific Blue anti-CD31 (Clone: WM-59), FITC anti-HLA-A,B,C (Clone: W6/32), and APC anti-HLA-DR (Clone: LN3) mAb (all from eBioscience). CIITA loss-of-function was identified by CD31+ cells that increased expression of HLA-A,B,C and not HLA-DR, with the positive threshold defined by fluorescence minus one staining with CD31 and HLA-A,B,C labeled cells. This gate was then used to collect CIITA^{null} EC using a 100 μm low-pressure nozzle on BD FACSAria II and then to deposit single cells into C-96 flat-bottom well plates containing either medium, medium supplemented with 10 μM ROCK-selective inhibitor Y-27632 (Sigma-Aldrich). After 24 h, cells were re-fed with fresh EGM-2/5% FBS medium that was changed every other day. Colonies were scored after 14 d and serially expanded into larger vessel sizes. To analyze phenotypic stability, HLA-DR^{neg} EC or unmodified EC were FACS isolated and expanded in EGM-2/5% FBS for 2 weeks. These cells were then challenged with TNF-α (10 ng/ml, Invitrogen) or IFN-γ (50 ng/ml) and harvested at 0, 1, 6, 12, and 24 h and stained with PE anti-E-selectin (CD62E, clone: 68-5H11, BD Pharmingen, San Jose, Calif.), FITC ICAM-1 (CD54, clone: LB-2, BD Pharmingen), PE PD-L1 (CD274, clone: MIH1, BD Pharmingen), FITC PECAM-1 (CD31, clone: WM-59, eBioscience) as well as APC HLA-DR (Clone: LN3, eBioscience), fluorescein labeled UAE-1 lectin (Vector Labs, Burlingname, Calif.), and eFluor 450 fixable viability dye (eBioscience) and analyzed on a LSR II flow cytometer (BD Biosciences, San Jose, Calif.) with post-acquisition analysis using FlowJo software (FlowJo LLC, Ashland, Oreg.). Unmodified and CIITA^{null} EC were also stained for VE-cadherin (CD144, clone: F-8, Santa Cruz Biotechnology, with secondary stain: goat anti-mouse IgG Alexa-488, Life Technologies) and mounted on slides using mounting medium (Prolong Gold; Invitrogen), and examined by microscopy with an Axiovert 200M microscope (Carl Zeiss, Thornwood, N.Y.).

qRT-PCR. RNA from EC was isolated using RNeasy Mini Kit (Qiagen, Valencia, Calif.) and used to make cDNA with the High-Capacity cDNA Reverse Transcription Kit (Applied Biosystems) per manufacturer's protocols. qRT-PCR reactions were assembled with TaqMan 2× Gene Expression Master Mix (Applied Biosystems) and pre-developed Taqman gene expression probes and analyzed on a CFX96 Real-Time system using CFX Manager Software (Bio-Rad, Hercules, Calif.). Probes used in this study were purchased from Applied Biosystems: GAPDH (Hs99999905_ml), CIITA (Hs00172094_ml), HLA-DRA (Hs00219575_ml), and CXCL10 (Hs01124251_gl). Gene expression levels were normalized to GAPDH.

PCR and Sanger Sequencing. Genomic DNA was isolated from clonally expanded EC using QuickExtract DNA Extract Solution (Epicentre, Madison, Wis.) according to the manufacturer's protocol. A 200bp segment containing the CIITA sgRNA target site was amplified by PCR using Platinum SuperMix High Fidelity (Invitrogen) using primers CIITA_forward: CACCAGCCCTCTTTCCAGA (SEQ ID NO: 5), CIITA_reverse: CCCCTTGCAATGATTTCTGT (SEQ ID NO: 6). The PCR amplicon was then column purified and subcloned into a TOPO TA vector using TOPO TA Cloning Kit (Invitrogen). Random colonies were picked and submitted for Sanger Sequencing using universal M13 forward and reverse sequencing primers at the W. M. Keck Sequencing Facility, Yale University. Same donor but unmodified EC were used as controls for comparison.

Mixed lymphocyte-endothelial reactions. Unmodified or CIITA^{null} ECs (1.5e5 cells/well) were plated into gelatin-coated wells of 24-well culture plates (Falcon; BD Biosciences) and treated with IFN-γ (50 ng/ml) (Invitrogen) where indicated. Purified memory CD4+ T lymphocytes were then added to each well (1.5e6 cells/well). All cultures were maintained in 5% CO2 at 37° C. The medium for co-culture consisted of RPMI 1640 supplemented with 10% FBS serum, 2 mM L-glutamine, 100 U/ml penicillin, and 100 μg/ml streptomycin (Invitrogen). Supernatants collected from cocultures were assayed using an ELISA kit for human IL-2 or IFN-γ (Platinum ELISA kits, eBioscience), according to directions provided by the manufacturer. To measure proliferation by CFSE dilution, lymphocytes were stained with 250 nM CF SE (Molecular Probes/Life Technologies) for 20 min before coculture with EC and collected after 7 d and stained with APC anti-CD4 (Clone: RPA-T4, eBioscience) mAbs and Pacific Blue anti-HLA-DR (Clone: LN3, eBioscience), and analyzed by flow cytometry. In some experiments, MHC II expression was restored by CIITA retrovirus as previously described (Manes T D, et al. The Journal of Immunology. 2007; 178:3237-3243). The retroviral supernatant was concentrated using 100 kD Amicon Ultra Centrifugal Filters (EMD Millipore, Billerica, Mass.)

Mice and Protein Gel Implants. All animal protocols were approved by the Yale Institutional Animal Care and Use Committee. Human microvessels were generated and implanted subcutaneously in the abdominal wall of female 6-8 week old C.B-17/SCID-beige mice (Taconic Biosciences, Germantown, N.Y.) as previously described (Shepherd B R, et al. The FASEB journal. 2006; 20:1739-1741). Briefly, ECFC-derived EC were suspended in a rat tail type I collagen gel and 400 ul of the cell suspension was gently poured into a single well of a 48-well tissue culture plate. The protein gel was polymerized at 37° C/5% CO₂ and then implanted. Each mouse received bilateral gel implants: one containing unmodified EC and the other containing CRISPR/Cas9 modified EC; mice were split into two cohorts defined by anatomic side of CIITA^{null} EC implant. Two weeks after implantation, animals were euthanized and the grafts harvested for analysis of human microvasculature. Gels and surrounding soft tissue were fixed in 10% neutral buffered formalin, embedded in paraffin, and 5 um thick sections were cut for H&E staining or immunostaining for human CD31 (BioGenex, Fremont, Calif.) and SM alpha-actin. Vessel number was quantified by number of perfused vessels, defined as containing murine erythrocytes, normalized to gel area and only vascularized implants were included for analysis. For challenge experiments, mice were given 400 ng recombinant human IFN-γ (Invitrogen) or PBS by subcutaneous injection every other day for an additional week. These gels were snap frozen in Tissue-Tek OCT (Sakura Finetek, Torrance, Calif.), cut in 5 um thick cryosections, and subsequently fixed in acetone and stained with fluorescein labeled UAE-1 and HLA-DR (Clone: LB3.1, with secondary stain: goat anti-mouse IgG DyLight-649, Vector Labs) for detection of HLA-DR⁺ human EC-lined vessels within engrafted protein gels on a fluorescent microscope (Axiovert). Investigators were not blinded to allocation of implants either during the experiment or when accessing outcomes. Previous experience with the collagen implant model was used to determine the number of animals needed to obtain statistical significance (Suárez Y, et al. The Journal of Immunology. 2007; 179:7488-7496). From this experience, 9 mice were implanted as approximately 80% of implanted collagen/EC matrices inosculate with the murine vasculature and at minimum 5-6 matrices with functional vessels are needed in each group for statistical comparisons.

Statistics. All data are expressed as mean±SD. Statistical comparisons were made using Student's t test or one-way ANOVA with Bonferroni post-hoc test as appropriate. P values of 0.05 or less were considered statistically significant. All results were computed using Prism v6.0 (GraphPad Software, Inc, La Jolla, Calif.).

The results of Example 1 experiments are now described.

Endothelial cell (EC) cultures from endothelial colony-forming cells (ECFC) have been extensively characterized and are essentially indistinguishable from human umbilical vein endothelial cells (HUVEC) with the single exception that they have a much greater replicative lifespan before displaying features of senescence. They are readily cultured from umbilical cord blood and, of particular relevance for this study, ECFC-derived EC display the same immunological properties as HUVEC isolated from the same umbilical cord. Lentiviral vectors were used to introduce tetracycline-inducible Cas9 and constitutively expressed RNA guide strands. Lentiviral transduction of EC is well tolerated, highly efficient (routinely exceeding 60% after a single round of infection), and the use of a tetracycline-inducible promoter to control Cas9 limits the exposure of the cells to possible accumulation of random off-target mutations by continuous overexpression of Cas9. Cloning conditions were optimized to routinely produce multiple different colonies with distinct bi-allelic deletions by the addition of a Rho-associated protein kinase (ROCK) selective inhibitor to microwells seeded with single EC.

### Transduction of Inducible Cas9 in ECFC-Derived EC

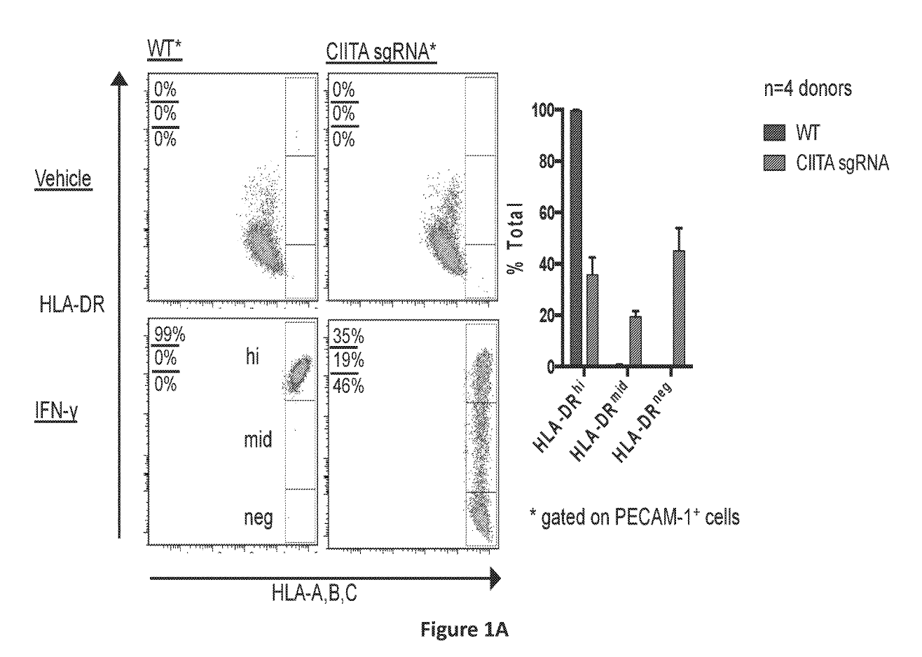

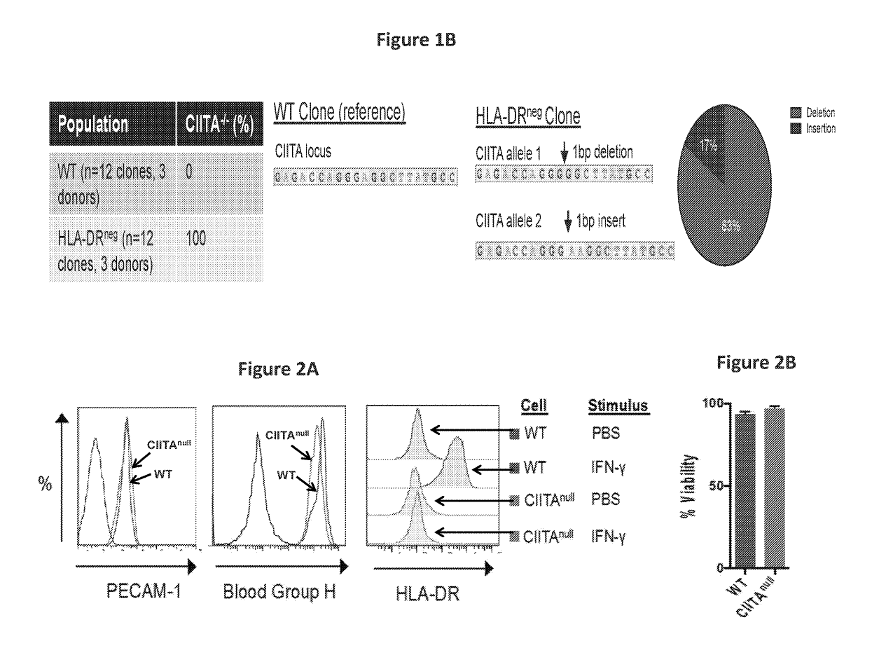

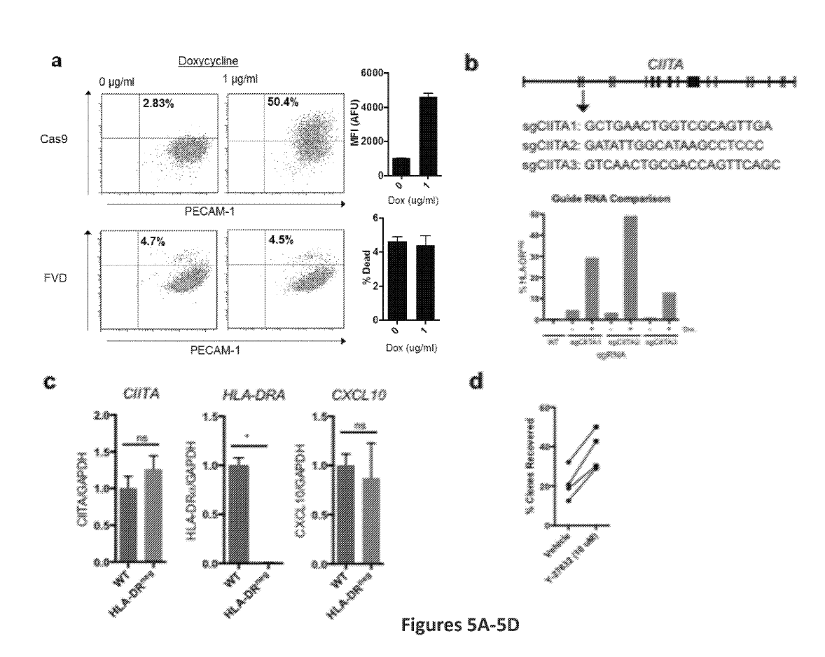

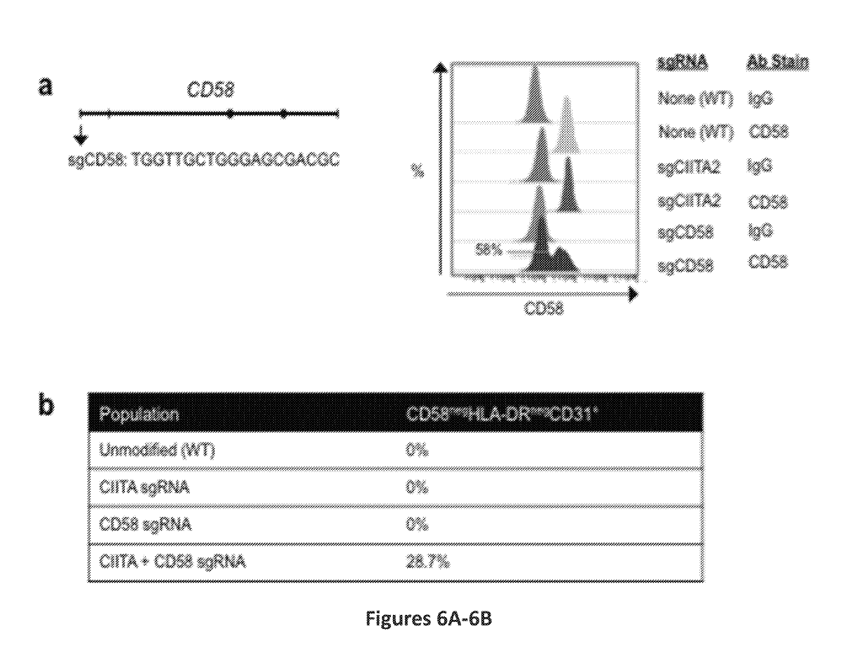

Early passage ECFC-derived human EC cultures were transduced with a tetracycline-inducible FLAG-tagged Cas9 lentiviral vector as described in the Methods. After a single round of transduction, over 95% of the cells remained viable and were FLAG-Cas9 negative in the absence of doxycycline. However, about 50% of the cells had detectable levels of Cas9 following doxycycline treatment (FIG. 5A) and the level of FLAG-Cas9 expression increased as a result of increased doxycycline up to 10 μgml. The cultures were then transduced with a second lentivirus that constitutively expressed an sgRNA designed to target an exonic region shared by all known splice variants of CIITA (FIG. 5B). Because CIITA is normally expressed at very low levels, even in interferon (IFN)-γ-treated EC, rather than assess its expression directly, its function was analyzed. Specifically, CIITA is an IFN-γ-inducible transactivator of class II MHC but not class I MHC molecule expression and identification of loss-of-function can be assessed through flow cytometric analysis of surface expression of class I MHC (HLA-A,B,C in humans) and class II MHC (HLA-DR being the most highly expressed form) before and after IFN-γ stimulation. While essentially 100% of unmodified EC upregulated both class I and II MHC molecules upon IFN-γ stimulation, delivery of CIITA-specific sgRNA followed by doxycycline treatment resulted in three distinct subpopulations differing in levels of induced class II MHC: HLA-DR^{hi}, which were indistinguishable from unmodified EC, HLA-DR^{mid}, which expressed reduced levels of MHC II, and HLA-DR^{neg}, which expressed no MHC II molecules (FIG. 1A). Because all three populations expressed equivalent levels of MHC I in response to IFN-γ stimulation, the HLA-DR^{neg} subpopulation likely represented EC with loss-of-function gene disruption of CIITA. This result was reproducible with several different donor EC cultures and with different CIITA-specific sgRNA sequences (FIG. 5B). CIITA mutagenesis was minimal in the absence of doxycycline, which is consistent with reduced levels of detectable Cas9 expression. To ascertain the generalizability of this approach, another gene, CD58, was targeted and a similar loss of expression was observed (FIG. 6A). Importantly, both genes were disrupted by simultaneously transducing the cultures with lentiviral constructs encoding different sgRNAs (FIG. 6B).

### Isolation and Characterization of CIITA^{null} EC

Cells that bore loss-of-function mutations in CIITA were detectable by surface staining, thus FACS sorting was used to isolate CRISPR/Cas9-modified HLA-DR^{neg} EC for further characterization. qRT-PCR analysis of FACS-sorted IFN-γ-stimulated unmodified EC and the HLA-DR^{neg} EC subpopulation revealed 99.2% reduction of HLA-DRA transcript in the latter, consistent with CIITA loss-of-function, but equivalent levels of transcripts for CIITA as well as CXCL10, another IFN-γ-stimulated gene. Individual clones were isolated to further characterize mutations of CIITA in HLA-DR^{neg} EC. FACS sorting was employed for single-cell isolation of EC and cloning efficiency was improved in HLA-DR^{neg} EC in cultures supplemented with the ROCK inhibitor Y-27632 (FIG. 5D). Clonally expanded HLA-DR^{neg} EC had a normal karyotype and could be grown for at least 10 passages after sorting before showing morphological evidence of cell senescence, similar to unmodified EC. After expansion, genomic DNA isolated from HLA-DR^{neg} EC clones was used to amplify a region of CIITA containing the CIITA-specific sgRNA target site. Consistent with loss-of-function, randomly selected HLA-DR^{neg} EC clones derived from three distinct donors were all confirmed to have bi-allelic indels at the predicted CIITA locus with a bias towards deletions as previously reported for CRISPR/Cas9 gene disruption (FIG. 1B).

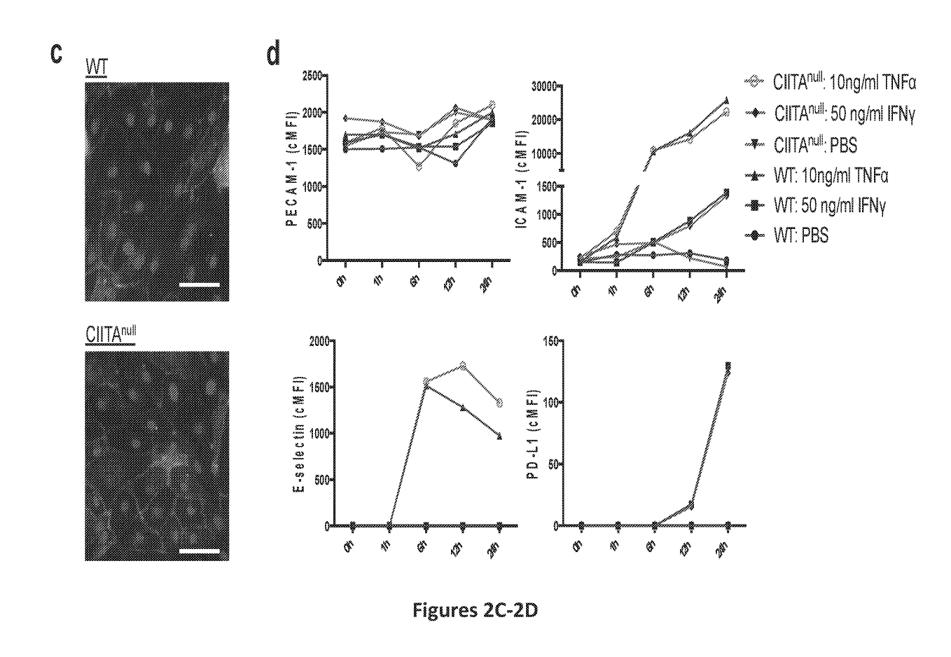

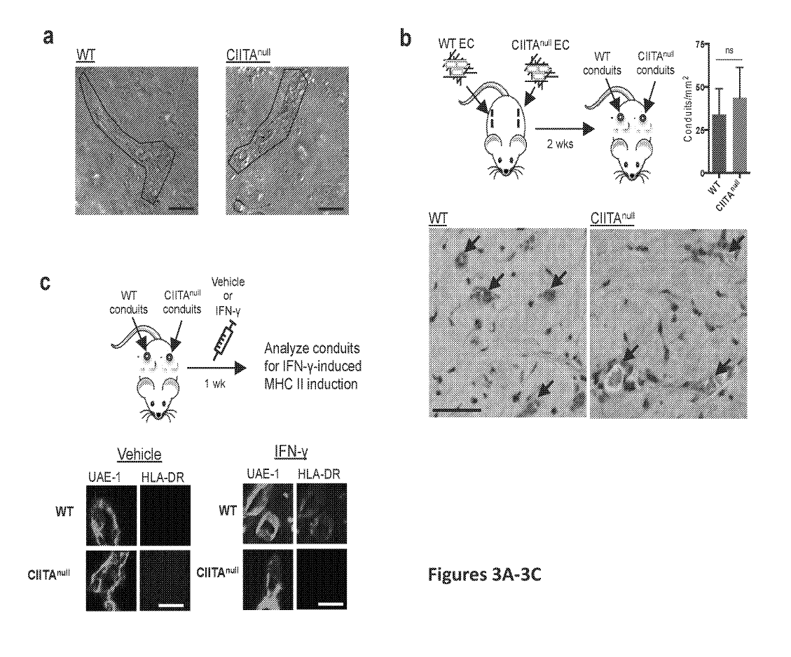

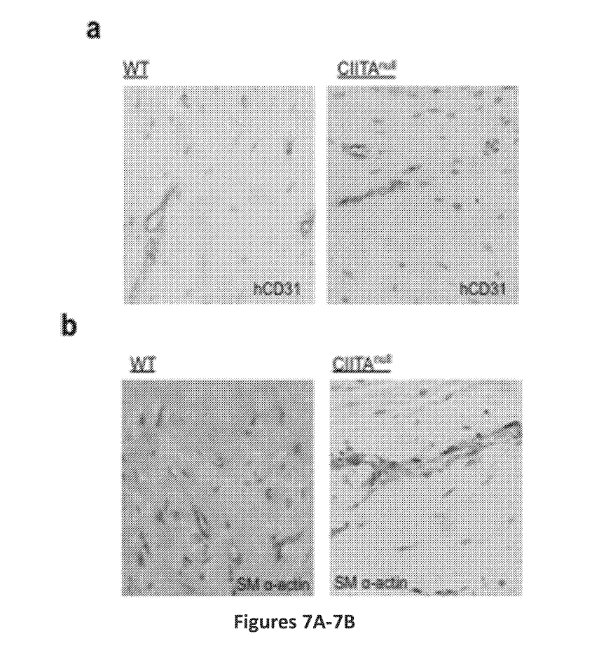

Having established that EC were efficiently produced by bi-allelic gene disruption, phenotypic functions of CIITA^{null} EC were then characterized. Serially passaged CIITA^{null} EC maintained high levels of expression of the EC markers PECAM-1 (CD31) and blood group H antigens, the latter detected with ulex europaeus agglutinin-1 (FIG. 2A), and formed VE-cadherin positive cell-cell lateral borders (FIG. 2C). Furthermore, while these cells were refractory to IFN-γ-induced expression of MHC II (FIG. 2A) they responded with similar kinetics and magnitude to TNF-α and IFN-γ induction of E-selectin, ICAM-1 and PD-L1 (FIG. 2D). When suspended and cultured in a 3-dimensional collagen matrix, CIITA^{null} EC spontaneously formed cords that underwent vacuolization, an early step of lumen formation (FIG. 3A), again in a manner indistinguishable from unmodified EC. CIITA^{null} EC suspended in collagen protein matrix and implanted subcutaneously into the abdominal wall of SCID/bg mice formed stable human EC-lined conduits that inosculated with host vessels, recruited murine smooth muscle alpha-actin positive supporting mural cells, and became perfused with murine blood (FIG. 3B and FIGS. 7A-7B). Comparison of the number of perfused conduits formed by CIITA^{null} EC to those from unmodified EC in the same host revealed no significant differences, suggesting CIITA-ablation in EC by CRISPR/Cas9 does not affect the intrinsic in vivo vessel-forming capability of these cells. Finally, consistent with in vitro results suggesting CIITA^{null} EC are refractory to IFN-γ-induced expression of MHC II, perfused conduits formed from unmodified EC expressed MHC II upon challenge with IFN-γ, whereas conduits derived from CIITA^{null} EC implanted in the same mouse but on the contralateral side did not (FIG. 3C).

### Immunogenic Function of CIITA^{null} EC

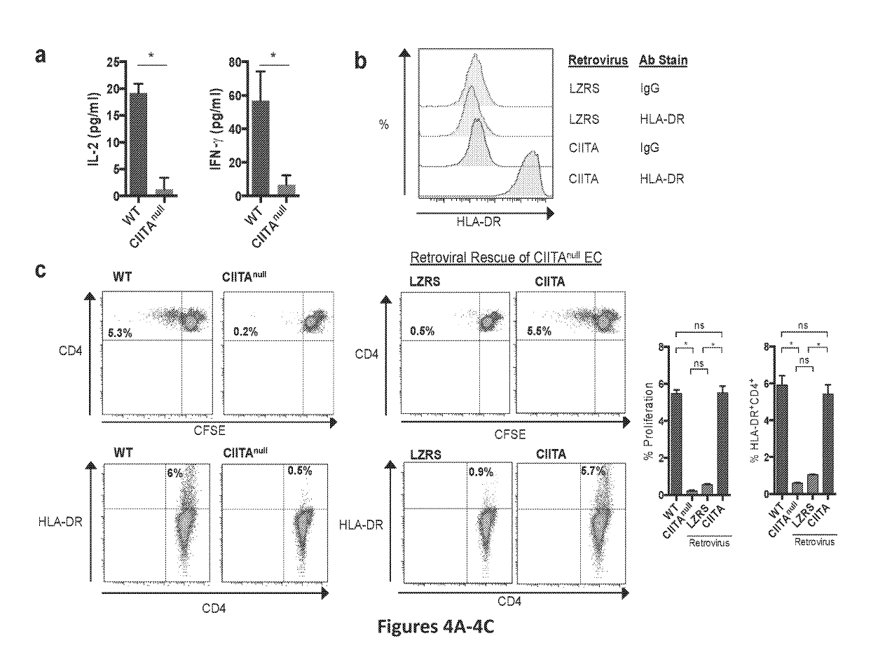

To demonstrate the utility of Cas9-mediated gene disruption in untransformed human EC, the immunological functions of the modified cells were analyzed. Immunological rejection of differentiated allogeneic cells is a major hurdle for therapeutic applications of ECFC-derived EC in regenerative medicine because human EC, unlike mouse EC, are capable of initiating allogeneic CD4+ T cell responses as a consequence of direct presentation of non-self forms of class II MHC molecules. In particular, co-culturing of IFN-γ-treated human EC with allogeneic CD4+ memory T lymphocytes results in T cell activation as indicated by expression of activation markers, including MHC II, cytokine production, and proliferation by the alloreactive subset. Consistent with CD4+ restriction to MHC II, the data herein demonstrate that ablation of CIITA by CRISPR/Cas9 in primary EC results in concomitant loss of the ability to activate alloreactive CD4+ memory T cells as measured by secretion of IL-2 and IFN-γ (FIG. 4A), and proliferation and expression/acquisition of MHC II on the alloreactive subset (FIG. 4C). In order to rule out off-target effects accounting for reduced EC immunogenicity, CIITA^{null} EC were transduced with a retrovirus expressing a wild-type copy of CIITA. Both MHC II expression (FIG. 4B) and the ability to activate CD4+ memory T cells were restored (FIG. 4C).

Studies examining the role human ECs play in regulating physiological and pathologic processes have extensively utilized primary human EC cell cultures. The range and power of this approach can be greatly extended by the application of genetic alteration using CRISPR/Cas9, but this has been difficult due to the limited replicative lifespan of such cells, the difficulty of their transfection, and their inability to be cloned. An approach to achieve high-efficiency gene disruption in primary EC using the CRISPR/Cas9 system is demonstrated herein. First, instead of the widely used HUVEC cultures, umbilical cord blood ECFC-derived EC were utilized. These cells have an increased replicative capacity and are otherwise indistinguishable from HUVEC, including the capacity to spontaneous self-assemble into vessels in vivo. Second, Cas9 coding sequences and guide strands were introduced by lentiviral transductions instead of inefficient plasmid transfection or excessive over-expression characteristic of adenoviral vectors. The availability of a single vector, tetracycline-inducible system, enabled temporally limiting the period of Cas9 expression and minimized accumulation of random off-target mutations after clonal isolation. Furthermore, CRISPR/Cas9 can readily be adapted for gene mutation or correction, which is not possible with shRNA. Third, transient exposure to a ROCK inhibitor was used to improve the efficiency of EC cloning. This step is necessary to obtain uniformly modified populations, especially when FACS sorting cannot be used for isolation of living cells, e.g. when the cell surface is unaffected by the genetic change. Cloning is particularly important if CRISPR/Cas9 is employed to alter rather than simply disrupt an endogenous gene.

To demonstrate the utility of the approach, CIITA, the master regulator of MHC II expression, was chosen, and activation of alloreactive CD4+ memory T lymphocytes, a biologically important capacity of human EC that has not been observed in mice, was interrogated. The relative ease with which a specific gene, or multiple genes, can be targeted for Cas9-mediated genomic perturbation provides an opportunity to study the effect of loss-of-function mutations in other EC regulated processes, including vasculogenesis, barrier maintenance, fibrinolysis, or leukocyte recruitment in ways that were not technically feasible before. While off-target effects remain a concern, this study temporally limited expression of Cas9 through use of a tetracycline-inducible promoter, used judicious selection of CIITA-specific sgRNA sequences, ascertained that other major EC functions were intact, and demonstrated phenotypic rescue by reintroduction of CIITA. Though off-target mutations at loci containing three or more mismatches are rare and often may not have functional consequences, single-cell cloning of ECFC-derived EC also permits screening for appropriate clones through PCR amplification and direct sequencing of putative off-target sites. The use of other Cas9 variants, including nickases or catalytically inactive variants fused to repressors or activators may also provide useful tools for human EC biology.

The intrinsic ability of human EC to self-assemble into vessels has been used to promote vasculogenesis in several pre-clinical models of ischemic injury, to tissue engineer vessel replacements, as well as to promote vascularization in larger bioengineered grafts. While these results are promising, allogeneic sources of EC may provoke cell-mediated immunological rejection. As demonstrated in this report, genetic ablation of CIITA eliminated surface class II MHC expression but did not compromise the ability of EC to self-assemble into vessels. The application of genome-editing technologies like CRISPR/Cas9 to modulate human cell behavior opens a range of exciting possibilities in regenerative medicine, including methods to reduce endothelial immunogenicity and allow the use of allogeneic EC as a cellular therapy or in tissue engineering.

In summary, a method for high efficiency gene disruption in untransformed human EC was developed in a manner that can be repeated to ablate expression of additional genes in the same cells. This approach may also be combined with the use of readily cultured human EC from ECFC, lentiviral transduction with CRISPR/Cas9 vectors, and enhanced cell cloning efficiencies to greatly expand the range of studies and applications that can be performed using human EC.

### Example 2

The materials and methods employed in Example 2 experiments are now described.

Isolation of human EC. Human umbilical vein endothelial cells (HUVEC) were isolated from umbilical cords following collagenase digestion and serially cultured on 0.1% gelatin-coated tissue culture plates in M199 (Invitrogen) supplemented with 20% FBS (Invitrogen), 1-glutamine (2 mM), penicillin (100 U/ml), streptomycin (100 μg/ml), 0.1% EC growth supplement, and porcine heparin (100 g/ml; Sigma-Aldrich). Human EC derived from endothelial colony-forming cells (HECFC) were differentiated from cord blood mononuclear cells in vitro, as previously described (Manes et al., J Immunol. 2007; 178(5):3237-3243). Briefly, umbilical cord blood was anticoagulated with heparin and enriched for mononuclear cells by density centrifugation using Lymphocyte Separation Medium (MP Biomedicals). These cells were then plated onto gelatin (0.1%, Avantor Performance Materials) and human-plasma fibronectin-coated (20 μg/ml, Millipore) tissue culture plates in EGM-2M media supplemented with 10 ng/ml VEGF (Lonza). Nonadherent cells were removed by washing after 4 days. Colonies of proliferating, differentiated cells were identified at 7-10 days, at which time the media was changed to EGM-2/15% FCS for expansion. HECFC-derived EC cultures were serially propagated in gelatin-coated tissue culture flasks with EGM-2/5% FBS (Lonza). After transduction with tetracycline-inducible Cas9 lentiviral vector, cultures were maintained in EGM-2 media using tetracycline-free FBS (Clontech).

siRNA knockdown. To silence CIITA, the following siRNA sequence was used: 5′-GAAGUGAUCGGUGAGAGUAUU-3′ (SEQ ID NO: 7) (ON-TARGETplus; Dharmacon, GE Healthcare). A nontargeting control sequence (Dharmacon, GE Healthcare) was used as a control. siRNA (25 nM) were introduced in EC using Lipofectamine RNAiMax (Invitrogen) according to manufacturer's instructions and then, at 24 hours, stimulated with human IFN-γ for approximately 48 hours and used for mixed-EC lymphocyte reactions, qPCR, or FACS analysis.

T_{EM} isolation. Human PBMC were collected with informed consent by leukapheresis from anonymized healthy volunteer donors under a protocol approved by the Yale Human Investigation Committee. Mononuclear cells were further enriched by density gradient centrifugation of leukapheresis products using Lymphocyte Separation Medium according to the manufacturer's protocol. Purified PBMCs were cryopreserved in 10% DMSO-90% FBS in liquid nitrogen before use. CD4⁺ and CD8⁺ T cells were isolated from PBMC using Dynabeads CD4⁺ and CD8⁺ Positive Isolation Kits (Invitrogen) per manufacturer's protocol. Naive, activated T cells, monocytes, and central memory T cells were removed by negative selection using anti-CD45RA (clone H100, eBioscience), anti-HLA-DR (clone LB3.1), anti-CCR7 (clone G043H7, BioLegend), and anti-CD62L mAb (clone DREG-56, eBioscience) at a concentration of 10 μg/ml for 20 minutes, washed twice, and magnetically depleted using pan-mouse IgG beads (Invitrogen). The population obtained by this procedure was routinely >95% HLA-DR⁻ CD45RA⁻CCR7⁻CD62L⁻CD45RO⁺CD4⁺ and HLA-DR⁻CD45RA⁻CRR7⁻CD62L⁻ CD45RO⁺CD8⁺ T_{EM} lymphocytes as analyzed by flow cytometry.

Flow cytometric analysis. To analyze EC with anti-CIITA, control siRNA treatment, or CRISPR/Cas9 ablation, HUVEC- or HECFC-derived EC were harvested with trypsin (TrypLE Express, Invitrogen), washed in 1× PBS, and subsequently stained with directly conjugated PB anti-PECAM-1 (clone WM-59), FITC anti-HLA-A, -B, -C (clone W6/32), FITC or APC anti-HLA-DR (clone LN3) (all from eBioscience), PE anti-GITRL (R&D Systems), FITC anti-ICAM-1(clone LB-2), PE anti-PD-L1 (clone MIH5), PE anti-CD40 (clone 5C3), FITC anti-LFA-3 (clone 1C3), PE anti-PD-L2 (clone MIH18), PE anti-OX40L (clone ik-1), or PE anti-ICOSL (clone 2D3) (all from BD Biosciences).

To analyze T lymphocytes, cells were collected after isolation or mixed EC-lymphocyte cocultures, washed in 1× PBS, and stained in FACS-staining buffer (PBS/1% BSA) with eFluor 450 fixable viability dye (eBioscience), anti-CD25 (clone BC96), PerCp-Cy5.5 anti-CD4 (clone RPA-T4), APC or PB anti-CD8 (clone SK1), PE anti-Granzyme B (clone GB11), PE or PB anti-Perforin (clone dG9,), FITC anti-CCR7 (clone G043H7), FITC anti-pSTAT5 (clone Y694), APC anti-CD62L (clone DREG56) (all from eBioscience), or PE anti-CD45RO (clone UCHL1, BioLegend). For experiments requiring intracellular staining (perforin, granzyme B, pSTAT5), cells were fixed in 2% paraformaldehyde and subsequently permeabilized (0.1% saponin/FACS buffer or BD Phosflow Perm Buffer III, BD Biosciences) before staining. Viability assays were performed according to manufacturer's instructions (eBioscience). All samples were analyzed on an LSR II flow cytometer (BD Biosciences) with postacquisition analysis using FlowJo software (FlowJo LLC).

Cytokine measurement by ELISA. Cell culture supernatants were collected from mixed EC-lymphocyte reactions at 24 hours and were assayed by sandwich ELISA for secreted human IL-2 or IFN-γ (Platinum ELISA kits, eBioscience), according to the manufacturer's instructions. CD8+ T_{EM} were cultured at 30:1 ratio with EC to detect elaborated IL-2 and IFN-γ.

Mixed EC-lymphocyte reactions. CD4+ T_{EM} activation by allogeneic EC requires recognition of nonself class II MHC molecules, principally HLA-DR, on the EC, which is lost on in vitro cultured cells. To reinduce EC expression of class II MHC, EC or HECFC-derived EC were treated with 50 ng/ml recombinant human IFN-γ (Invitrogen) for 48 hours.

For in vitro stimulation, EC were washed with HBSS, plated to confluence in gelatin-coated 24-well plates, and then 1×10⁶ allogeneic CD8⁺ T_{EM} and/or 1×10⁶ allogeneic CD4⁺ T_{EM} were added to each well in RPMI 1640 (Invitrogen), supplemented with 10% FBS (Invitrogen), 1-glutamine (2 mM), penicillin (100 U/ml), and streptomycin (100 μg/ml). T cells were labeled with 250 nM CFSE (Molecular Probes, Invitrogen) prior to coculture and assessed at 7 days for CFSE dilution. In some experiments, EC were pretreated with anti-CIITA or control siRNA prior to IFN-γ treatment. Where indicated, cocultures were supplemented 25 U/ml recombinant human IL-2 (BioLegend), 1:1 dilution of CD4+ T_{EM} conditioned medium, or 10 μg/ml of blocking anti-HLA-DRα or irrelevant isotype control were added into allogeneic cocultures. For transwell experiments, CFSE-labeled CD8+ T_{EM} (10:1 ratio) were cultured in a transwell insert (0.4-μM pore size, Corning Inc.) above a in C-24 well monolayer of CD4⁺ T_{EM} and EC (10:1 ratio) that were either pretreated with anti-CIITA or control siRNA.

For generation of conditioned medium, EC were retrovirally transduced with the extracellular domain of Fcγ receptor IIA (CD32), as previously described (Manes et al. J Immunol. 2007; 178(5):3237-3243). CD32⁺ EC were then overlayed with 2.5 μg/ml of isotype control (Mouse IgG2_{a,k,} BioLegend) or anti-CD3 (clone OKT3, BioLegend) for 4 hours, washed, and then purified CD4⁺ T_{EM} were added. After 24 hours, medium was collected, and excess antibody was removed by protein G-coated beads (Invitrogen) for 1 hour at 4° C. on continuous shaking and then frozen for subsequent use or analysis by ELISA. In some experiments, IL-2 was immunoabsorbed from the activated CD4⁺ T_{EM} conditioned with 20 μg/ml anti-IL-2 (clone AB12-3G4, eBioscience) and subsequently immunoabsorbed with protein G beads, resulting in specific depletion of IL-2 below limits detectable by ELISA.

For cytotoxicity assays, EC were plated onto C-96 U-well microtiter plates (Corning) and after 24 hours, approximately 200×10³ allogeneic CD8⁺ T_{EM} were added to each well. At 72 hours, RPMI medium, activated CD4⁺ T_{EM} conditioned medium, activated CD4⁺ T_{EM} conditioned medium depleted of IL-2, control conditioned medium, or control conditioned medium with 25 U/ml IL-2 (eBioscience) were added at 1:1 dilution to the wells. At 7 days, CD8⁺ T_{EM} were collected and restimulated with fresh EC containing 1:1 medium conditions. Approximately 72 hours after replating, additional medium conditions were added. At 14 days, cells were collected, purified by CD8+isolation, and assayed by FACS or for cytotoxicity as described previously (Kreisel et al. J Immunol. 2004; 173(5):3027-3034, Kummerow et al. Eur J Immunol. 2014; 44(6):1870-1872). Briefly, target EC were preloaded with 500 nM calcein AM (Molecular Probes, Invitrogen) and CD8+T_{EM} were added in AIMV medium supplemented 10 mM HEPES (Invitrogen) at a 30:1 ratio and assayed for calcein release on a fluorescent plate reader (BioTek) in bottom reading mode at 37° C. for 4 hours. The percentage of cytotoxicity was defined as (live−experimental)/(live−lysed)×100, where live is the values of calcein-loaded EC without CTL, experimental is with CTL, and lysed is lysed with 1% Triton-X 100 (Sigma-Aldrich).

Arterial transplantation. Arterial transplantation was performed as previously described (Lorber et al., Transplantation. 1999; 67(6):897-903). In brief, 1- to 3-mm segments of diameter-matched human epicardial coronary arteries harvested from explanted human hearts of organ donors or recipients were interposed into the infrarenal aortae of female C.B-17 SCID/bg mice (Taconic Biosciences) by end-to-end microsurgical anastomotic technique. Adjacent human artery segments were transplanted in groups of 2-5 mice for each experiment, and data from individual experiments were pooled for analysis. Approximately 2-7 days after transplantation, 2×10⁸ human PBMC allogeneic to the artery graft were adoptively transferred into mice i.p. As previously described, only T and B lymphocytes are successfully engrafted by this procedure, and only T cells appear in the circulation (Lorber et al., Transplantation. 1999; 67(6):897-903). Successful engraftment, achieved in all mice in this study, was defined as detection by flow cytometry of a distinct population of human CD3⁺ cells, ranging from 0.5%-10% of the total circulating mononuclear cells.

In vivo HLA-DR blockade. For HLA-DR blockade experiments, injecting intact blocking HLA-DR antibodies depleted mice of circulating human T cells. To overcome this problem, F(ab)′2 fragments from this antibody were prepared using a F(ab)′2 preparation kit (Thermo Scientific) per manufacturer's instructions. Mice bearing human artery grafts were injected s.c. with 200 μg loading and 100 μg every 48 hours maintenance doses of anti-HLA-DRα (clone LB3.1) or with irrelevant IgG control (Jackson ImmunoResearch Laboratories Inc.) starting on the day before allogeneic PBMC transfer. After 21 days of treatment, animals were anesthetized and human arterial grafts were perfused with normal saline and excised before death. Arterial grafts were snap frozen in optimum cutting temperature compound (OCT compound), and serial 5-μm transverse sections were cut for morphometric, immunofluorescence, and qPCR analyses.

CRISPR/Cas9-mediated ablation of CIITA in EC. CIITA^{null} HECFC-derived ECs were generated as previously described (Abrahimi et al., Circ Res. 2014; 117(2):121-128). Briefly, early-passage HECFC were transduced with lentiviral constructs encoding doxycycline-inducible Cas9 and CIITA specific guide sequence (GATATTGGCATAAGCCTCCC) (SEQ ID NO: 2). After 48 hours, cells were drug selected and Cas9 expression was induced with doxycycline and blasticidine treatment for 7 days and then stimulated with 50 ng/ml IFN-γ. CIITA loss-of-function EC were identified by FACS gated on HLA-DR⁻cells, isolated using a 100-μm low-pressure nozzle on BD FACSAria II, then expanded and used for qPCR, in vitro mixed EC-lymphocyte reactions, and formation of synthetic vessels.

Synthetic microvessel formation and transplantation. Human microvessels were generated and implanted s.c. in the abdominal wall of female 6- to 8-week-old C.B-17 SCID/bg mice as previously described (Shepherd et al., FASEB J. 2006; 20(10):1739-1741). Briefly, HECFC-derived EC were suspended in a rat tail type I collagen gel, and 400 μl of cell suspension was gently poured into a single well of a 48-well tissue culture plate. The protein gel was polymerized at 37° C./5% CO₂ and then implanted. Each mouse received a single implant containing either unmodified EC or CRISPR/Cas9-modified CIITA^{null} EC. Approximately 14 days after implantation, WT or CIITA^{null} gel implanted mice were distributed into three groups categorized by inoculation: no PBMC (PBS), PBMC, or with PBMC depleted of CD8⁺ cells. In the PBMC group, approximately 2×10⁸ human PBMC allogeneic to the EC graft were adoptively transferred into mice i.p. In the CD8⁺-depletion group, 2×10⁸ human PBMC were depleted of CD8⁺ with Dynabeads CD8⁺ beads and then adoptively transferred. Approximately 10 days after inoculation, animals were euthanized and grafts were harvested for analysis of human microvasculature. Gels and surrounding soft tissues were fixed in 10% neutral buffered formalin and embedded in paraffin, and 5-μm thick sections were cut for H&E staining or immunostaining for human CD31 (BioGenex). Vessel number was quantified by number of perfused vessels (containing murine erythrocytes) normalized to gel area, and only vascularized implants were included for analysis. Previous experience with the collagen implant model indicated that n=3-5 per group are needed to obtain statistical significance (Suarez et al., J Immunol. 2007; 179(11):7488-7496).

Histology and immunofluorescence. Cross sections (5-μm) of artery grafts were stained with Elastica van Gieson (EVG) as well as H&E and used to quantify the luminal and intimal areas using ImageJ (NIH) for morphometry. The intimal area was defined as bound by internal elastic lamina and the lumen. The EC lining of grafts were identified by Rhodamine ulex europaeus agglutinin 1 (Vector Laboratories) and/or CD31 expression, which yielded equivalent results, and quantified by tracing the circumference of lumen using Image.” Graft-infiltrating human lymphocytes were quantified by staining for human CD45RO⁺ (BD Biosciences) or CD3⁺ (Dako), which yielded equivalent results, by indirect immunofluorescence (donkey anti-rabbit or donkey anti-mouse AlexaFluor-594, Invitrogen). The numbers of intimal CD45RO⁺ were counted in two cross sections per graft. All sections were mounted on slides using mounting medium (Pro-Long Gold, Invitrogen) and examined by microscopy with an Axiovert 200M microscope (Zeiss).

qPCR analysis. RNA from cultured EC was isolated using RNeasy Mini Kit (QIAGEN) and used to make cDNA with the High-Capacity cDNA Reverse Transcription Kit (Applied Biosystems) per manufacturer's protocols. To isolate RNA from artery grafts, serial sections of flash-frozen artery were immersed briefly in water, centrifuged, and resuspended in RLT lysis buffer and then isolated by RNeasy kit. qPCR reactions were assembled with TaqMan 2× Gene Expression Master Mix (Applied Biosystems) and predeveloped Taqman gene expression probes and analyzed on a CFX96 Real-Time system using CFX Manager Software (Bio-Rad). Probes used in this study: GAPDH (Hs02758991_g1), HPRT1 (Hs02800695_m1), CIITA (Hs00172094_m1), HLA-DRA (Hs00219575_m1), HLA-DPA1 (Hs01072899_m1), HLA-DPB1 (Hs03045105_m1), HLA-DQA1 (Hs03007426_mH), HLA-DQB1 (Hs03054971_m1), CD3e (Hs01062241_m1), CD4 (Hs01058407_m1), CD8 (Hs00233520_m1), PRF1 (Hs00169473_m1), GZMB (Hs00188051_m1), CXCL10 (Hs01124251_g1), IFN-γ (Hs00989291_m1), and IL-2 (Hs00174114_m1), all from Applied Biosystems. Gene expression levels were normalized to GAPDH, HPRT, CD3e, or CD8 where indicated.

Statistics. All data are expressed as mean±SD. Statistical comparisons were made using Student's t test (2-tailed) or 1-way ANOVA with Bonferroni post-hoc test. P values less than 0.05 was considered statistically significant. All results were analyzed with Prism v6.0 (GraphPad).

Study approval. All human cells (cord blood, PBMC), tissues (umbilical cords), and vessels were obtained with informed consent under protocols approved by the Yale Human Investigation Committee and the New England Organ Bank. All animal protocols were approved by the Yale Institutional Animal Care and Use Committee.

The results of Example 2 experiments are now described.

### MHC Class II Blockade Reduces Allogeneic T Cell-Mediated Injury and CD8⁺ CTL-Associated Transcripts in Human Arterial Grafts In Vivo

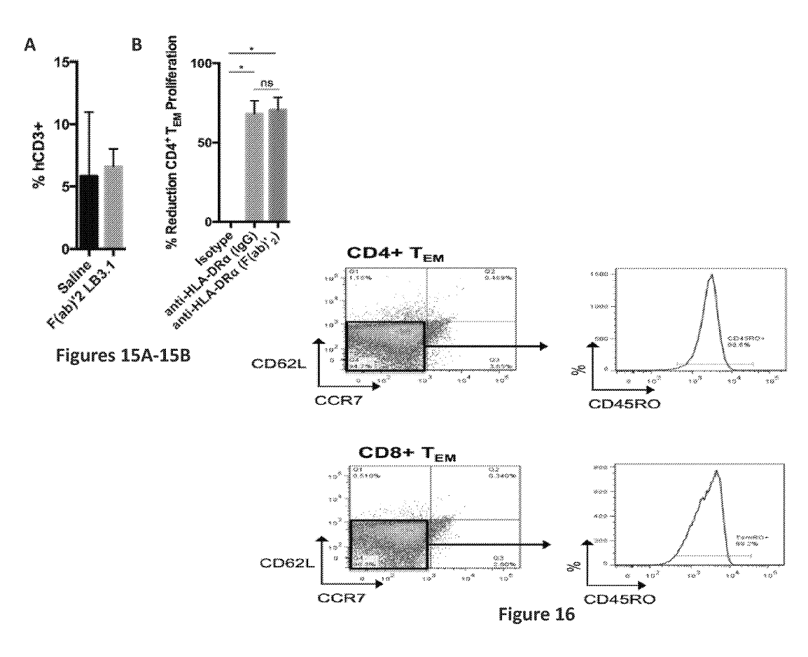

The contribution of graft EC expression of class II MHC molecules was investigated in a model of human intimal arteritis/endothelialitis. A small segment of human epicardial coronary artery was grafted end-to-end into the infrarenal aorta of an immunodeficient C.B-17 SCID/bg mouse. Subsequent adoptive transfer of human peripheral blood mononuclear cells (PBMC) allogeneic to the artery donor led to engraftment of circulating CD4⁺ and CD8⁺ T lymphocytes, but not neutrophils, monocytes, DC, or natural killer cells (Lorber et al., Transplantation. 1999; 67(6):897-903, Shultz et al., Nat Rev Immunol. 2007; 7(2):118-130). Over the next 3-4 weeks, circulating human T cells infiltrated the intima of the human artery graft and destroyed the luminal EC but generally spared the vessel media. Adjacent human coronary artery grafts were implanted from the same donor into pairs of mice, following which recipient mice were adoptively transferred with human PBMC allogeneic to the artery donor and given either F(ab)′2 fragments of mouse anti-HLA-DRα or mouse IgG control for 21 days. HLA-DR was selectively targeted because, even though human EC also express HLA-DP and HLA-DQ molecules, HLA-DR is the dominant stimulator of allogeneic T cell responses (Hirschberg et al., Immunol Rev. 1982; 66(1):57-77). F(ab)′2 fragments were used because intact anti-HLA-DRα antibody reduced the numbers of human T cells present in the circulation following adoptive transfer, whereas F(ab)′2 fragments blocked CD4+ T cell activation by EC in vitro but did not reduce circulating human T cell numbers in vivo (FIG. 15A-15B).

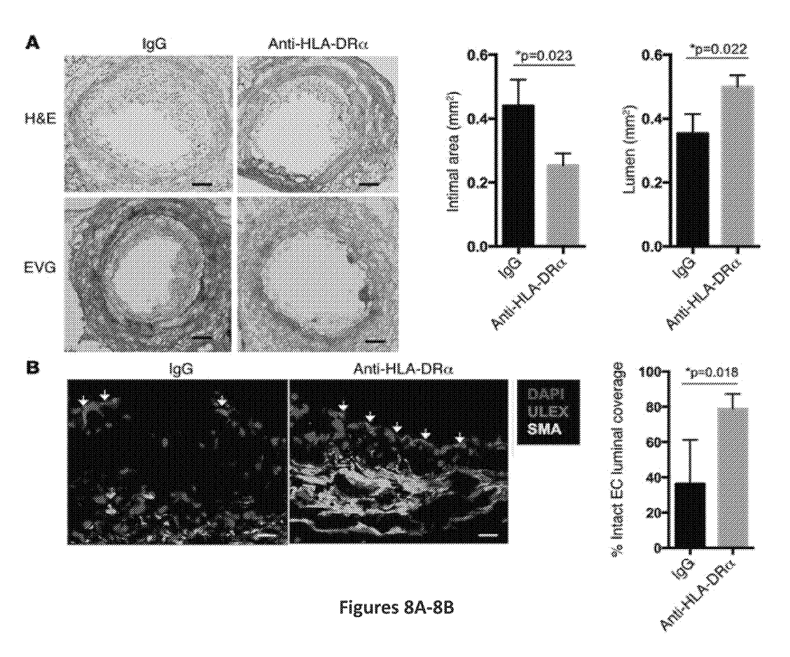

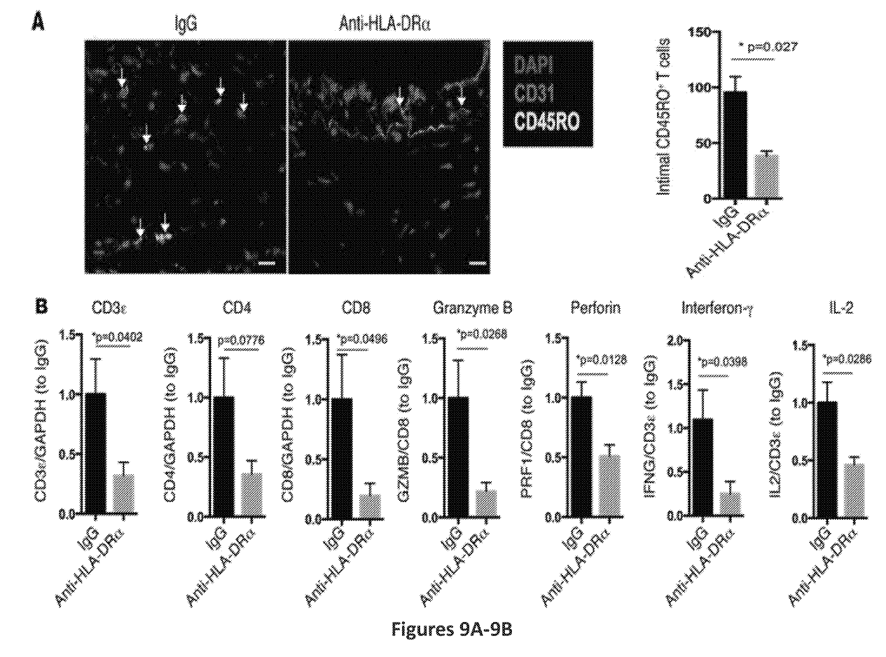

Blocking HLA-DR recognition significantly reduced the intimal area and luminal narrowing and preserved the human EC lining of the graft compared with control F(ab)′2 treatment (FIGS. 8A-8B). There was also a significant reduction in the total T cell infiltration and cytokine transcripts within the intima of anti-HLA-DRα F(ab)′2-treated animals (FIGS. 9A-9B). Despite the fact that this anti-HLA-DR antibody does not prevent CD8⁺ T cells from interacting with class I MHC molecules, there was a decrease in perforin and granzyme B transcripts when normalized to a CD8-specific marker (FIG. 9B), consistent with inhibition of CTL development. Collectively, these data support the conclusion that recognition of class II MHC molecules on the EC in this model promotes acute CTL-mediated rejection, consistent with the working hypothesis that activation of CD4+ T_{EM} in response to class II MHC molecules expressed by EC is needed to provide help for development of CD8⁺ T_{EM} into effector CTL. Protection was incomplete, perhaps because only HLA-DR was blocked and not HLA-DP and -DQ, which are also expressed by human EC, albeit at lower levels. Furthermore, despite preserving circulating T cell numbers, a more subtle effect of the antibody on T cell function cannot be ruled out. These limitations of antibody-mediated effects led to the exploration of genetic approaches to human immunology both in vitro and in vivo.

Silencing Class II MHC Expression in EC Directly Inhibits CD4⁺ but not CD8⁺ T_{EM} Responses In Vitro

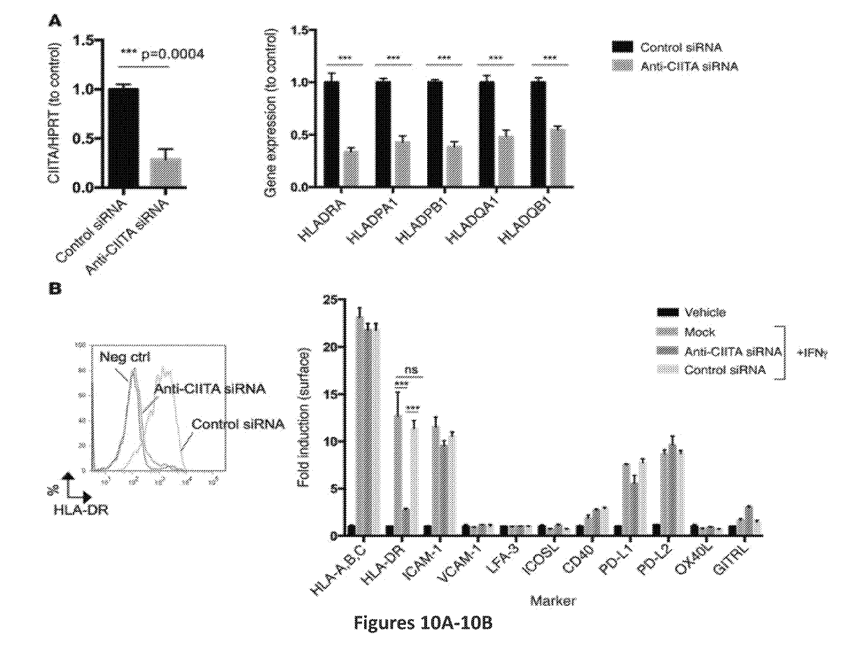

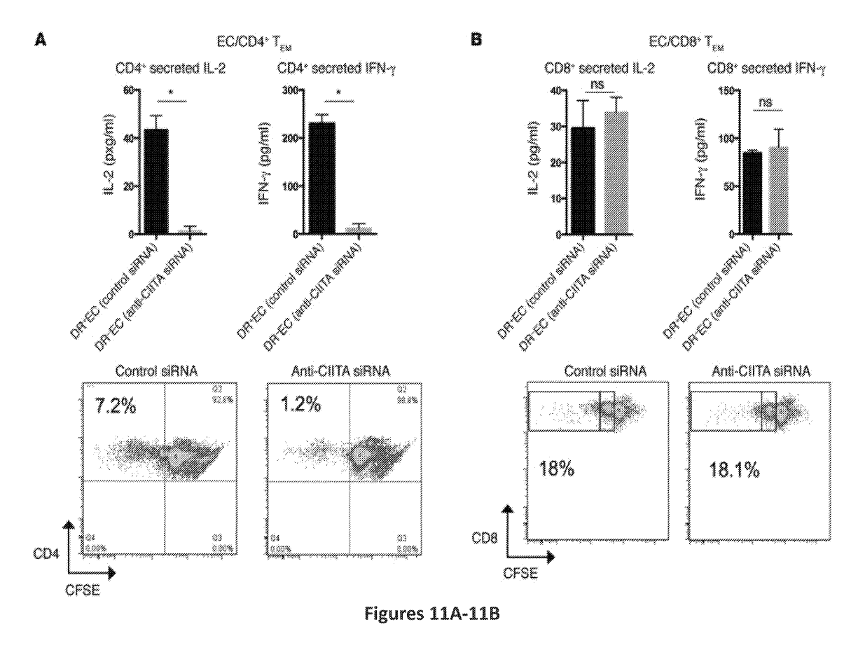

Human CD4⁺ and CD8⁺ T_{EM} interactions with EC were modeled using mixed EC-lymphocyte reactions in vitro. Class II MHC molecule expression was initially reduced with an siRNA specific for the class II MEW transactivator (CIITA). Knockdown of CIITA inhibited the IFN-γ-dependent induction of CIITA and blocked transcription at all three class II MHC loci (HLA-DR, -DP, and -DQ) as detected by quantitative PCR (qPCR) (FIG. 10A). Consequently, HLA-DR surface protein was effectively reduced (FIG. 10B). Treatment with anti-CIITA siRNA did not affect expression of class I MEW or other immunologically relevant adhesion or costimulatory molecules (FIG. 10B). CD4⁺ T_{EM} (CD45RO⁺CCR7⁻CD62L⁻CD4⁺) isolated from human PBMC (FIG. 16) and cocultured with allogeneic EC, sequentially pretreated with anti-CIITA siRNA and IFN-γ, secreted significantly less IL-2 and IFN-γ and proliferated less when compared with CD4⁺ T_{EM} cocultured with sequential control siRNA and IFN-γ-treated EC (FIG. 11A). The same comparison showed no differences in IL-2 or IFN-γ secretion or in proliferation (FIG. 11B) by CD8⁺ T_{EM} (CD45RO⁺CCR7⁻CD62L⁻CD8⁺). These data show that changes observed within the vessel wall in vivo by anti-HLA-DR antibody are likely due to inhibition of CD4⁺ T cell recognition of class II MHC on graft EC.

EC-Activated CD4⁺ T_{EM} Enhance CD8⁺ T_{EM} Responses to Allogeneic EC

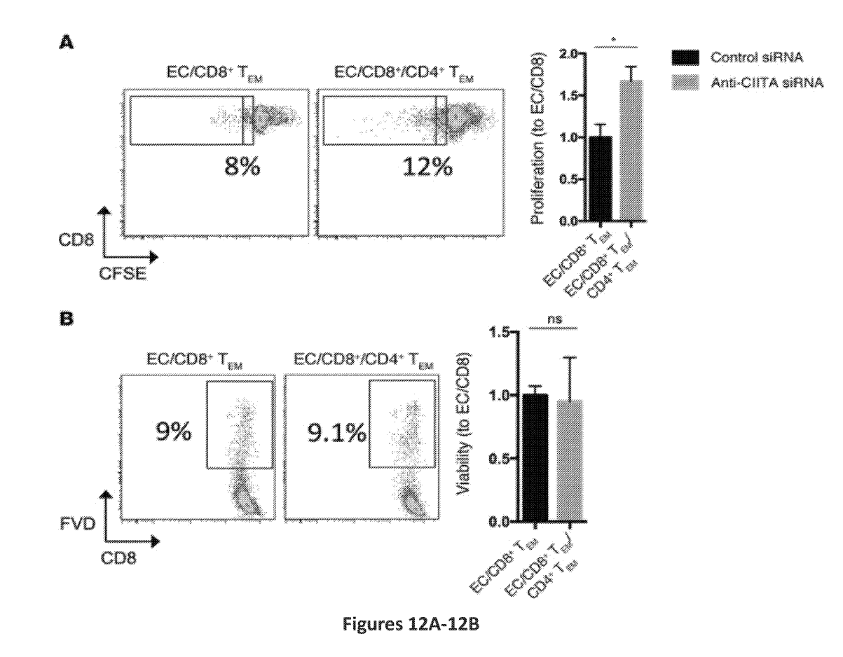

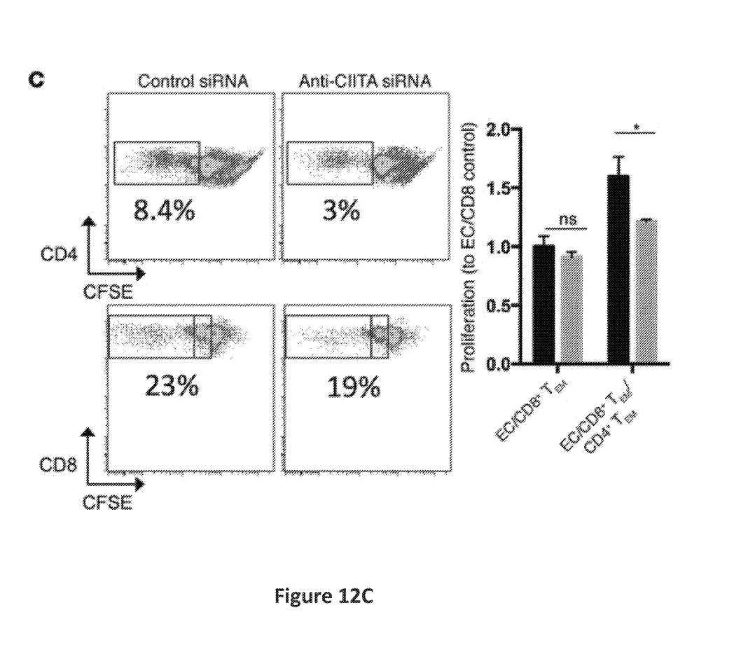

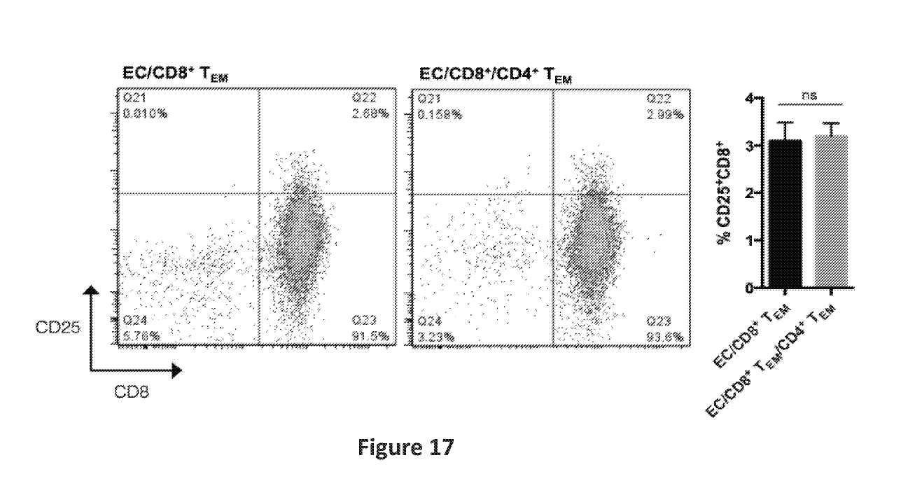

To determine if CD4⁺ T_{EM} activated by EC can provide help for CD8⁺ T_{EM}, the effect of adding CD4⁺ T_{EM} to EC-CD8⁺ T_{EM} mixed cocultures was examined. The addition of CD8⁺ T_{EM} enhanced CD8⁺ T_{EM} proliferation (FIG. 12A). In the absence of CD8⁺ T_{EM}, proliferation typically stopped after a single division, whereas the presence of CD4⁺ T_{EM} promoted additional rounds of division indicated by further dilution of CFSE dye. These differences were not due to changes in TCR engagement of MHC class I alloantigens, as addition of CD4⁺ T_{EM} did not significantly change the number of CD8⁺ T_{EM} cells that initially upregulated CD25, the inducible component of the high-affinity IL-2 receptor and an early activation marker that reflects TCR engagement (FIG. 17). The enhanced proliferation was also not due to increased survival of CD8⁺ T_{EM} cells, as there was no significant difference in viability at 7 days with or without CD4⁺ T_{EM} present (FIG. 12B). To determine if CD4⁺ T_{EM} enhancement of EC-reactive CD8⁺ T_{EM} required concomitant activation of CD4⁺ T_{EM} by the allogeneic EC, CD4⁺ and CD8⁺ T_{EM} were cocultured with allogeneic EC that were pretreated with anti-CIITA siRNA to uncouple MHC class II-dependent activation of CD4⁺ T_{EM} from MHC class I-dependent activation of CD8+T_{EM}. The increase in proliferation by CD8⁺ T_{EM}, due to the addition of CD4+ T_{EM} to the cultures, was attenuated by pretreatment of the EC with anti-CIITA siRNA (FIG. 12C).This result implies that CD4⁺ T_{EM} must first be activated by allogeneic EC before they can help CD8⁺ T_{EM}.

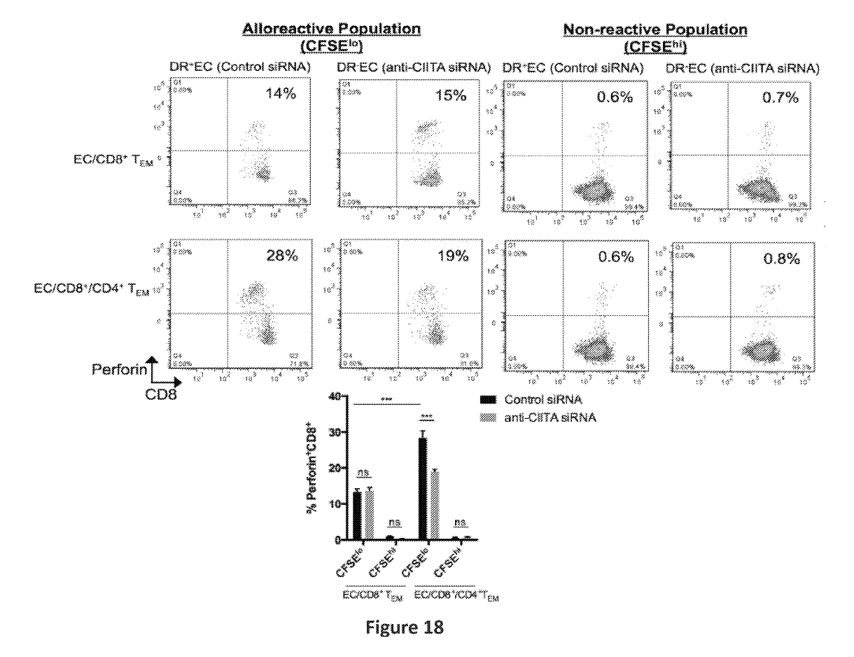

The phenotype of EC-reactive CD8⁺ T_{EM} was further analyzed using their dilution of CFSE to separate the reactive (CFSE^{lo}) and nonreactive (CFSE^{hi}) populations. Perforin expression appeared restricted to the CFSE^{lo}CD8⁺ T_{EM} population, and the number of perforin⁺CFSE^{lo}CD8⁺ T cells increased with the addition of CD4⁺ T_{EM} (FIG. 18). Inhibiting MHC class II expression on allogeneic EC attenuated the enhancement of perforin expression in the reactive population. In contrast, few cells in the nonreactive CD8⁺ T_{EM} population expressed perforin, and this was unchanged by the addition of CD4⁺ T_{EM} with or without pretreatment of the EC with anti-CIITA siRNA (FIG. 18). These results show that EC-reactive CD4⁺ T_{EM} promote perforin expression, a marker of CTL maturation, within the CD8⁺ T_{EM} population.

CD4⁺ T_{EM} Provide Help to CD8⁺ T_{EM} Via Paracrine Provision of IL-2

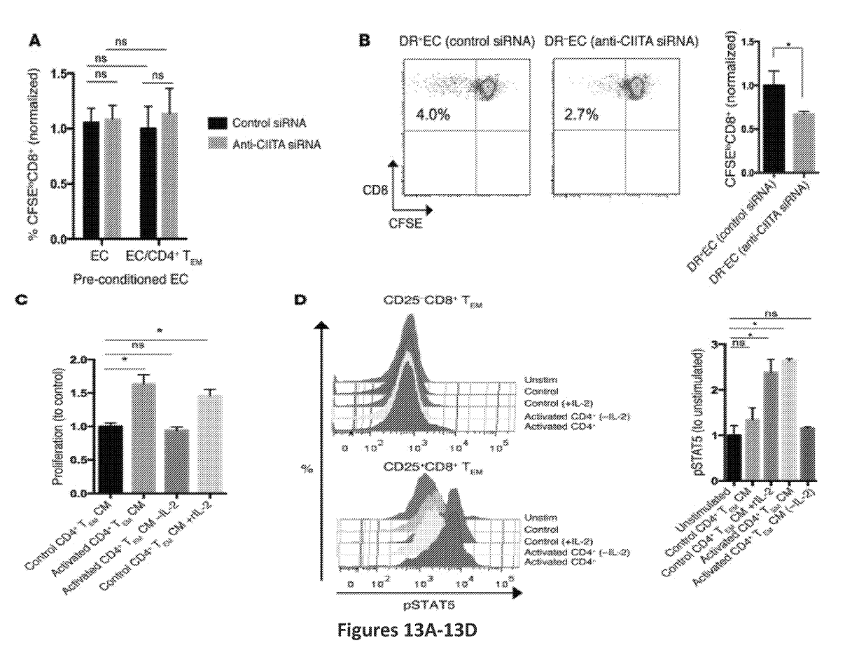

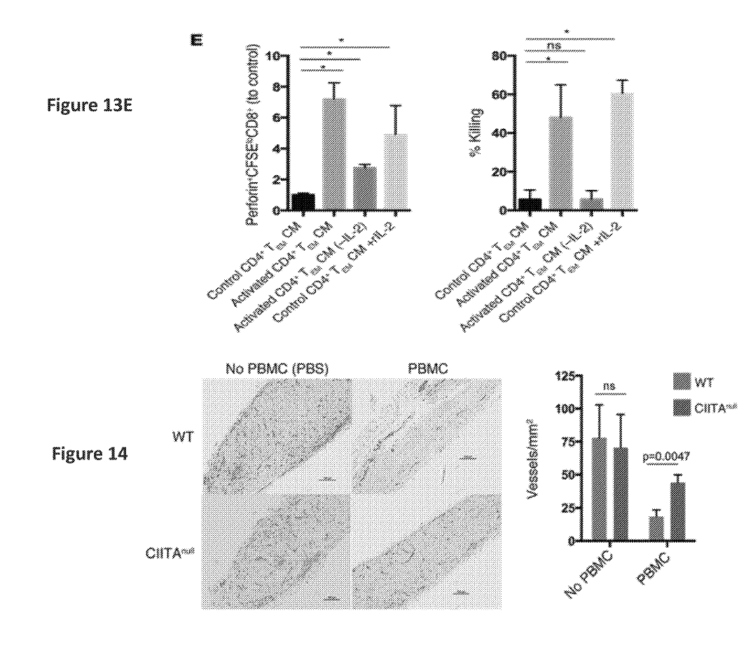

Possible mechanisms by which allogeneic EC-activated CD4⁺ T_{EM} help CD8⁺ T_{EM} activation and CTL development were investigated, including: (i) modifying (licensing) EC to become more immunogenic to CD8⁺ T_{EM} (e.g., upregulation of EC MHC class I, costimulatory molecules, and/or transpresentation of cytokines such as IL-15); (ii) enhancing CD8⁺ T_{EM} responses in a contact-dependent manner (e.g., upregulation of CD4⁺ T cell costimulatory molecules like CD40L facilitating T-T interactions); or (iii) enhancing CD8⁺ T_{EM} in a contact-independent manner (e.g., CD4⁺ T cell paracrine release of IL-2 or other signals). To determine if CD4⁺ T_{EM} license EC (Ridge et al., Nature. 1998; 393(6684):474-478), EC cultures were pretreated with either anti-CIITA or control siRNA, activated with IFN-γ, cocultured CD4⁺ T_{EM}, and FACS isolated for subsequent culture with CD8⁺ T_{EM}. No significant difference was observed in CD8⁺ T_{EM} alloresponses between control EC or EC preconditioned with CD4⁺ T_{EM} (FIG. 13A), ruling out any functional changes in EC immunogenicity toward CD8⁺ T_{EM}. To examine if CD4⁺ T_{EM}-driven enhancement of EC-reactive CD8⁺ T_{EM} responses is contact dependent, CD4⁺ and CD8⁺ T_{EM} cocultures were separated with EC by a semipermeable transwell membrane. CD4⁺ T_{EM} cocultured with control siRNA/IFN-γ-treated EC were able to enhance the CD8⁺ T_{EM} response compared with CD4⁻ T_{EM} cocultured with EC pretreated anti-CIITA siRNA/IFN-γ, even across a semipermeable membrane, providing evidence for the existence of a soluble “helper” factor (FIG. 13B).

To identify the potential factor(s) responsible for help in this assay, conditioned medium was generated by coculturing CD4⁺ T_{EM} with EC transduced to express an Fc receptor (FcR; CD32) and overlaying with anti-CD3 mAb. In this procedure, T cells are polyclonally activated in the context of costimulators expressed by human ECs, enhancing the generation of soluble mediators. After 24 hours of primary coculture, conditioned medium from EC-activated CD4⁺ T_{EM} or control medium was collected and used in EC-CD8⁻ T_{EM} cocultures. Consistent with the results generated by transwell experiments, activated CD4⁻ T_{EM}-conditioned medium enhanced proliferation of EC-reactive CD8⁺ T_{EM} (FIG. 13C). Since IL-2 is produced by activated CD4⁺ T_{EM} and has previously been shown to enhance CD8⁺ responses to allogeneic EC (Biedermann et al., J Immunol. 1999; 162(12):7022-7030), the contribution of IL-2 to the effects observed with conditioned medium was tested. Removal of IL-2 by immunoabsorption neutralized the effect of the conditioned medium, and addition of IL-2 to the control medium replicated the effect of activated conditioned medium. STATS is a transcription factor that is known to control cell cycle progression in effector T cells and is phosphorylated with IL-2-, IL-7-, or IL-15-initiated common γ chain cytokine signaling. Phosphorylation of STATS was observed in CD25⁺ but not CD25⁻CD8⁺ T_{EM} with activated conditioned medium, and this response was abrogated with IL-2 immunoabsorption (FIG. 13D). Perforin expression was increased when CD8⁺ T_{EM} were cocultured with EC in the presence of CD4⁺ T_{EM} conditioned medium, again in only the EC-reactive (CFSE^{lo}) population (FIG. 6E). This response was also largely abrogated with IL-2 immunoabsorption. Similarly, when adapted to a CTL killing assay of allogeneic EC, CD8⁺ T_{EM} expanded with the same allogeneic EC either in the presence of activated CD4⁺ T_{EM} conditioned medium or the exogenous IL-2, which both demonstrated significant cytotoxicity (FIG. 6E).

### Genetic Ablation of CIITA in Synthetic Blood Vessels Limits T Cell-Mediated Destruction In Vivo

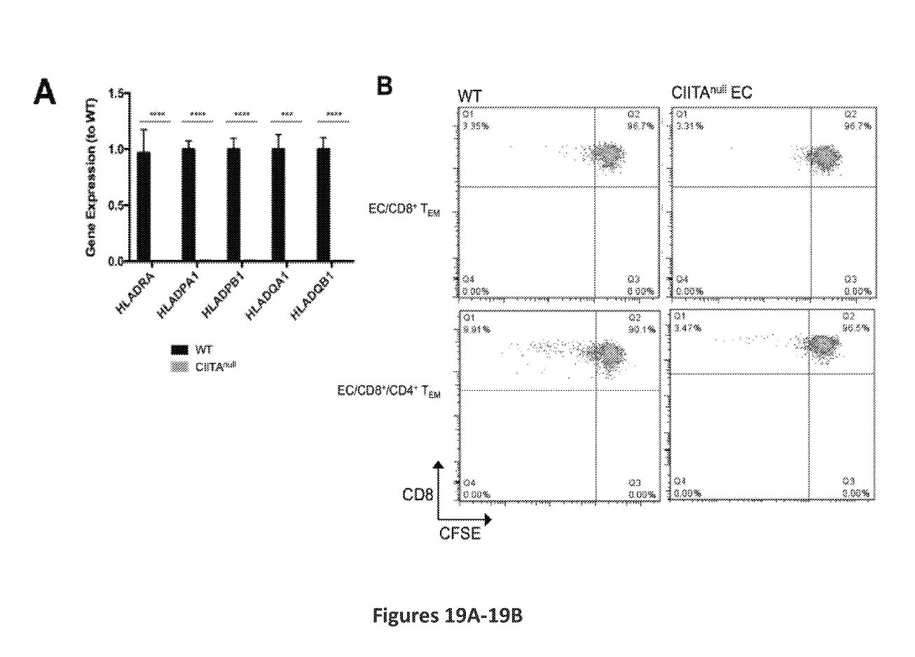

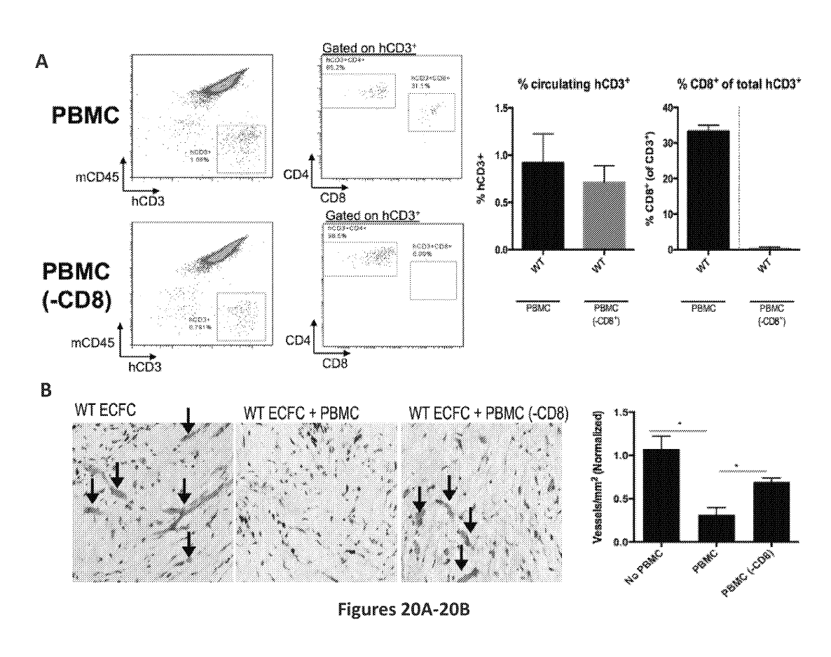

The genetic approach was extended to confirm the effects of EC-mediated activation of CD4⁺ T cells on cell-mediated rejection in vivo. siRNA effects are generally too short-lived and shRNA effects are too incomplete for effective use over the timeframe required to study graft rejection in vivo. A method was developed to genetically modify untransformed human EC derived from endothelial colony-forming cells (HECFC, also known as human cord blood EC) using CRISPR/Cas9 to specifically ablate CIITA. Inactivating mutations in CIITA prevented IFN-γ-induced class II MHC molecule expression, but not class I MHC molecule expression, and reduced allogeneic CD4⁺ memory T cell responses (Abrahimi et al., Circ Res. 2014; 117(2):121-128). Importantly, CIITA^{null} EC otherwise retained phenotypic functions of unmodified cells, including the ability to form synthetic microvascular beds when implanted into C.B-17 SCID/bg mice. CD4⁺ T_{EM} enhanced CD8⁺ T_{EM} responsiveness to HECFC-derived EC in vitro and the effect was attenuated by CIITA ablation due to effects on class II MHC molecule expression (FIG. 19A-19B), recapitulating results observed with siRNA treatment of human umbilical vein EC (HUVEC). When synthetic vessels are formed from human EC following implantation into C.B-17 SCID/bg mouse hosts and then challenged with adoptive transfer of human PBMC allogeneic to the EC, the human EC-lined microvessels are largely destroyed over a period of 10 days (Zheng et al., J Immunol. 2004; 173(5):3020-302639, Suárez et al., J Immunol. 2007; 179(11):7488-7496). Experiments using alloreactive T cell lines generated against cultured human EC suggested this effect is likely mediated by direct recognition of alloantigen on the EC (Zheng et al., J Immunol. 2004; 173(5):3020-302639). To determine if this model could be used to quantify CTL-mediated killing using PBMC, experiments using selective adoptive transfers were conducted. Adoptive transfer of CD8⁺ T cells was inefficient in the absence of CD4⁺ T cells, but CD4⁻ T cells readily engrafted in the absence of CD8⁺ T cells (Murray et al., Am J Pathol. 1998; 153(2):627-638). Inoculation of mice with unfractionated PBMC led to the appearance of both human CD4⁺ and CD8⁺ T cells in the circulation, whereas PBMC depleted of CD8⁺ cells prior to adoptive transfer produced a circulating human T cell population consisting wholly of CD4⁺ T cells (FIG. 20A). The absence of CD8⁺ T cells sharply reduced the destruction of synthetic vessels at 10 days following adoptive transfer, despite similar numbers of circulating human T cells, implying that most of the graft destruction is CD8⁺ T cell-mediated (FIG. 20B). Synthetic human EC-lined vessels were engineered using either unmodified (WT) or CIITA^{null} EC and then human PBMC allogeneic to the EC were adoptively transferred to determine if CD4⁺ T cell activation played any role in this CD8⁺ T cell response. The destruction of synthetic microvessels formed from CIITA^{null} EC was markedly attenuated compared with WT EC (FIG. 14). There was still some destruction of microvessels formed from CIITA^{null} EC in the absence of CD8⁺ T cells (82%±20% of control, n=3 mice), suggesting that some rejection may not involve direct recognition of EC MHC molecules. However, the difference between vessel loss following adoptive transfer of whole or CD8⁺-depleted PBMC was ablated (WT: 75%±12%, 82%±20%, n=3 mice each), suggesting that in this in vivo model, recognition of allogeneic class II MHC molecules on EC by human CD4⁺ T cells is once again required for provision of CD4⁺ T cell help for human CD8⁺ T cell activation and efficient expansion into CTL. As these animals do not have functional secondary lymphoid organs, this process, as in the human artery graft model, likely takes place within the graft itself.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A modified endothelial cell comprising a nucleic acid capable of downregulating gene expression of a gene selected from the group consisting of a class I and class II major histocompatibility complex (MHC) molecule, CD58, CIITA, NRLC5, beta2 microglobulin, ICOS-ligand, 4-1BB ligand, O×40 ligand, GITR ligand, interleukin 1 alpha, interleukin 6, ICAM-1, ICAM-2, VCAM-1, E-selectin, P-selectin and combinations thereof, and wherein the modified endothelial cell has reduced immunogenicity in an allogeneic environment.

2. The modified endothelial cell of claim 1, wherein the nucleic acid capable of downregulating gene expression is selected from the group consisting of a siRNA and a CRISPR system.

3. The modified endothelial cell of claim 2, wherein the siRNA comprises SEQ ID NO:7.

4. The modified endothelial cell of claim 2, wherein the CRISPR system further comprises a Cas expression vector and a guide nucleic acid sequence specific for the gene.

5. The modified endothelial cell of claim 4, wherein the guide nucleic acid sequence comprises a single guide RNA.

6. The modified endothelial cell of claim 5, wherein the single guide RNA comprises at least one sequence selected from the group consisting of SEQ ID NO: 1, SEQ ID NO: 2, SEQ ID NO: 3, and SEQ ID NO: 4.

7. The modified endothelial cell of claim 4, wherein the Cas expression vector is under the control of an inducible promoter.

8. The modified endothelial cell of claim 4, wherein the Cas expression vector is a viral vector selected from the group consisting of a Sendai viral vector, an adenoviral vector, an adeno-associated viral vector, a retroviral vector, and a lentiviral vector.

9. A modified endothelial cell comprising an inducible Cas9 expression vector; a single guide RNA specific for CD58; and a single guide RNA specific for CIITA, wherein induction of Cas9 expression results in the cell having reduced immunogenicity in an allogeneic environment.

10. A method for generating a modified endothelial cell comprising:
culturing endothelial colony forming cells (ECFCs); and
introducing a nucleic acid capable of downregulating gene expression of an endogenous gene selected from the group consisting of a class I and class II major histocompatibility complex (MHC) molecule, CD58, CIITA, NRLC5, beta2 microglobulin, ICOS-ligand, 4-1BB ligand, O×40 ligand, GITR ligand, interleukin 1 alpha, interleukin 6, ICAM-1, ICAM-2, VCAM-1, E-selectin, P-selectin and combinations thereof in the ECFCs,
wherein the modified endothelial cell has reduced immunogenicity in an allogeneic environment.

11. The method of claim 10, wherein the ECFCs are obtained from cord blood endothelial progenitor cells.

12. The method of claim 11, wherein culturing the ECFCs comprises differentiating cord blood mononuclear cells.

13. The method of claim 10, wherein introducing the nucleic acid further comprises introducing a nucleic acid capable of downregulating gene expression selected from the group consisting of a siRNA and a CRISPR system.

14. The method of claim 13, wherein the siRNA comprises SEQ ID NO:7.

15. The method of claim 13, wherein the CRISPR system comprises a Cas expression vector and a guide nucleic acid sequence specific for a gene, and introducing the nucleic acid comprises introducing the Cas expression vector and the guide nucleic acid into the ECFCs; and
inducing Cas expression from the Cas expression vector and, wherein the expressed Cas interacts with the guide nucleic acid sequence to mutate a loci for the gene.

16. The method of claim 15, wherein introducing the Cas expression vector comprises transducing the ECFCs with a viral Cas expression vector.

17. The method of claim 16, wherein the viral Cas expression vector is selected from the group consisting of a Sendai viral vector, an adenoviral vector, an adeno-associated viral vector, a retroviral vector, and a lentiviral vector.

18. The method of claim 15, wherein inducing the Cas expression vector comprises exposing the ECFCs to an agent that activates an inducible promoter in the Cas expression vector.

19. The method of claim 15, wherein the guide nucleic acid sequence is a single guide RNA.

20. The method of claim 19, wherein the single guide RNA comprises at least one sequence selected from the group consisting of SEQ ID NO: 1, SEQ ID NO: 2, SEQ ID NO: 3, and SEQ ID NO: 4.

21. Use of the modified endothelial cell of claim 1 in the manufacture of a medicament for regenerating vascular structures in a subject in need thereof.

22. Use of the modified endothelial cell of claim 1 in the manufacture of an engineered tissue or organ.

23. A pharmaceutical composition comprising the modified endothelial cell generated according to the method of claim 10 and a pharmaceutically acceptable carrier.

24. An engineered tissue or organ comprising the modified endothelial cell of claim 1.

25. A method for promoting vascular self-assembly in a subject in need thereof comprising administering to a subject an effective amount of a modified endothelial cell capable of forming vascular structures, wherein the modified endothelial cell comprises a nucleic acid capable of downregulating gene expression of an endogenous gene selected from the group consisting of a class I and class II major histocompatibility complex (MHC) molecule, CD58, CIITA, NRLC5, beta2 microglobulin, ICOS-ligand, 4-1BB ligand, O×40 ligand, GITR ligand, interleukin 1 alpha, interleukin 6, ICAM-1, ICAM-2, VCAM-1, E-selectin, P-selectin and combinations thereof, and wherein the modified endothelial cell has reduced immunogenicity in an allogeneic environment while maintaining the capacity to form vascular structures.

26. A method for regenerating vascular structures in a subject in need thereof comprising administering to a subject an effective amount of a modified endothelial cell capable of forming vascular structures, wherein the modified endothelial cell comprises an inducible Cas9 expression vector; a guide nucleic acid sequence specific for CD58; and a guide nucleic acid sequence specific for CIITA, and wherein induction of Cas9 expression results in the modified endothelial cell having reduced immunogenicity in an allogeneic environment while maintaining the capacity to form vascular structures.

27. Use of the modified endothelial cell of claim 9 in the manufacture of a medicament for regenerating vascular structures in a subject in need thereof.

28. Use of the modified endothelial cell of claim 9 in the manufacture of an engineered tissue or organ.

29. An engineered tissue or organ comprising the modified endothelial cell of claim 9.
